(19) United States
(12) Patent Application Publication
(10) Pub. No.: US 2026/0020816 A1
(43) Pub. Date: Jan. 22, 2026

(54) ASSESSMENT APPARATUS AND ASSESSMENT METHOD FOR OBJECTIVE PAIN ASSESSMENT

(71) Applicants: SHANGHAI JIAO TONG UNIVERSITY, Shanghai (CN); SHANGHAI REACOOL MEDICAL TECHNOLOGY CO., LTD., SHANGHAI (CN)

(72) Inventors: Ke MA, Shanghai (CN); Yue ZHANG, Shanghai (CN); Chunhui QIN, Shanghai (CN)

(21) Appl. No.: 19/258,294

(22) Filed: Jul. 2, 2025

(30) Foreign Application Priority Data
Jul. 16, 2024 (CN) 2024109530581
Mar. 21, 2025 (CN) 2025103504145

(51) Int. Cl. A61B 5/00; A61B 5/372

(52) U.S. Cl. CPC A61B 5/4824; A61B 5/372; A61B 5/7257; A61B 5/7267

(57) ABSTRACT

An assessment apparatus and method for objective pain assessment are provided, wherein the apparatus includes a processor comprising a frequency-domain transformation module, a frequency-band segmentation module, a first assessment submodule, and a second assessment submodule. The frequency-domain transformation module generates a global time-frequency feature matrix, which is segmented by the frequency-band segmentation module in a frequency domain into five frequency bands associated with pain perception. The first assessment submodule extracts last time step features and an adjacency matrix from the time-frequency feature matrix and generates a first feature vector representing global association patterns among electrodes used for acquiring EEG signals. The second assessment submodule generates a second feature vector with local spatiotemporal dynamic features of EEG signals, concatenates it with the first feature vector to form a fused feature vector, computes its class probability distribution, normalizes it, and generates an objective pain quantification indicator corresponding to the EEG signals.

This application claims priority to Chinese Patent Application No. CN 2024109530581 filed on Jul. 16, 2024 and to Chinese Patent Application No. CN 2025103504145 filed on Mar. 21, 2025, which are hereby incorporated by reference as if fully set forth herein.

### FIELD

The present disclosure generally relates to the technical field of pain assessment, and more particularly to an assessment apparatus and assessment method for objective pain assessment.

### BACKGROUND

Chronic pain refers to unpleasant sensory and emotional experience associated with actual or potential tissue damage, or resembling such experiences. Recognized as the fifth vital sign, pain is as important as respiration, blood pressure, pulse, and body temperature, and is a key indicator for assessing and monitoring human health. As the fifth vital sign, pain holds deeper significance in that it reflects the warmth of humanistic care in the evolution of medical practice. Its inclusion transforms the observation of vital signs from simple collection of physiological data into a profound care and attentiveness toward life.

Nevertheless, pain, as the fifth vital sign, faces an inherent limitation, namely the absence of objective biomarkers. Currently, pain assessment mainly relies on self-assessment of subjects and professional judgment from clinicians based on different scales, including the Visual Analogue Scale (VAS), the Numerical Rating Scale (NRS), as well as the DN4 and IDpain questionnaires for neuropathic pain. One significant drawback of these scale-based assessment methods lies in their reliance on subjective reporting, with a lack of objective markers.

Existing practice of pain assessment is typically based on the subjective judgment of either subjects or clinicians. For example, the subject is asked to select a number within a range of 0 to 100 to represent the perceived severity of pain, with a score from 0 to 39 indicating mild pain, 40 to 69 indicating moderate pain, and 70 to 100 indicating severe pain. This subjective scoring method prevents clinical pain assessments from reflecting actual clinical conditions, thus significantly hindering proper formulation of treatment plans, assessment of therapeutic effectiveness, and even medico-legal assessments.

Electroencephalography (EEG) equipment is currently one of the primary tools used in pain research. EEG records brain signals that reflect voltage fluctuations generated by neuronal firing. Recent studies have shown that voltage changes in different brain regions are associated with different types and intensity levels of pain. However, these voltage changes cannot accurately reflect the severity of pain experienced by subjects. In addition, EEG equipment is expensive and involves complex data processing, requiring signal acquisition to be conducted in specialized hospitals. Although recent scientific studies have demonstrated certain advantages of EEG in assessing pain intensity, it has yet to become an objective measure for pain assessment due to limitations such as signal acquisition challenges and the need for expert analysis.

US20010037222A1 discloses a system and method providing accurate, quantifiable and reproducible pain assessment documentation utilized in pain management. In particular, the pain assessment system can be implemented as follows. A description input mechanism prompts the collection of patient pain episode data, and a pain assessment mechanism assesses the pain episode data for the patient. A pain score generation mechanism then generates a multidimensional pain score from the pain episode data to quantify a pain condition for the patient. The method can be broadly summarized by the following steps: acquiring pain episode data for the patient; performing pain assessment for the patient; and generating a multidimensional pain score that quantifies a pain condition for the patient. However, due to inter-individual differences in perception, the classification in this technical scheme fails to achieve objective quantification of pain intensity and thus fails to provide an objective pain index. For instance, in certain pain scenarios, neural signals may be exaggerated or suppressed by noise, in turn affecting the accurate quantification of pain intensity. Meanwhile, pain perception is inherently dynamic, yet a model lacking a temperature parameter processes all inputs on a fixed scale and therefore fails to capture real-time changes in the functional connectivity, such as differences in neural activity patterns observed during pain escalation or alleviation. This technical solution cannot adaptively adjust for inter-individual differences in neural signals, resulting in inaccurate quantification of pain intensity in certain populations, such as individuals with low neural sensitivity or with different pain thresholds.

As discussed previously, the existing pain detection methods in the existing art are not suitable for objective assessment of pain intensity. How to establish an objective pain assessment apparatus, model, and method—particularly a pain assessment apparatus, model, and method in which the pain index evaluation process is objective and unaffected by inter-individual perceptual differences, thereby meeting objectivity requirements—remains an unresolved issue.

In view of this, the present disclosure provides a new calculation method, which constructs a “digital twin pain brain” by leveraging model-based validation and iterative fitting. The system collects data with mature EEG devices and inputs the computational results into a calculation model. By utilizing the digital twin pain brain model for computation and comparison, it ultimately presents an objective numerical indicator. The disclosed method is the first approach to unifying pain, as the fifth vital sign, with other vital signs such as body temperature and pulse in a visualized, objective, and quantifiable manner, thereby forging a new path to objective pain assessment.

### SUMMARY

To address the shortcomings of the prior art, the present disclosure provides, in a first aspect, an assessment apparatus for objective pain assessment, comprising a processor. The processor includes a frequency-domain transformation module, a frequency-band segmentation module, a first assessment submodule, and a second assessment submodule. The frequency-domain transformation module performs fast Fourier transform on pain-related EEG signals to generate a global time-frequency feature matrix. The frequency-band segmentation module segments the global time-frequency feature matrix in a frequency domain into band-specific time-frequency feature matrices of five frequency bands associated with pain perception, namely δ, θ, α, β, and γ. The first assessment submodule is configured to extract time-series features of the EEG signals from the band-specific time-frequency feature matrices, extracts last time step features and an adjacency matrix from the time-series features, and generates a first feature vector representing global association patterns among electrodes used for acquiring EEG signals based on the last time step features and the adjacency matrix. The second assessment submodule performs convolution on the time-series features to generate a second feature vector containing local spatiotemporal dynamic features of the EEG signals, concatenates the first and second feature vectors along a feature dimension to form a fused feature vector containing both global association patterns and local spatiotemporal dynamic features, computes a class probability distribution of the fused feature vector, performs normalization, and generates an objective pain quantification indicator corresponding to the EEG signals.

The frequency-domain transformation module of the present disclosure converts EEG signals into a global time-frequency feature matrix using fast Fourier transform (FFT) and dynamically defines frequency bands associated with pain perception (δ, θ, α, β, γ) using a learnable filter set in the frequency-band segmentation module. The common methods that establish frequency bands in a fixed manner cannot adapt to frequency-band differences among individuals (e.g., age-related shift of the α frequency band). By contrast, the disclosed scheme herein adaptively adjusts frequency-band boundaries via end-to-end training of filter parameters, accurately matching individualized spectral characteristics. The cross-frequency-band attention mechanism fuses energy distribution and phase coupling characteristics across different frequency bands, revealing pain-related neural oscillation coordination patterns and providing physiologically meaningful fundamental features for objective quantification.

The first assessment submodule extracts long-term dependencies in the time series using a bidirectional gated recurrent unit (BiGRU) and employs a Gumbel-Softmax unit to generate a dynamic adjacency matrix. A graph aggregation unit produces a first feature vector representing global association patterns among electrodes used for acquiring EEG signals, overcoming the limitations of common predefined brain network templates. This approach captures real-time changes in functional connectivity strength among brain regions induced by pain (e.g., transient activation of the thalamo-insular pathway). Meanwhile, the second assessment submodule extracts local high-frequency oscillatory features (e.g., transient γ-band responses in the anterior cingulate cortex (ACC)) using a convolutional neural network. Fusing global brain-network features and local spatiotemporal features comprehensively represents the neurodynamic process of pain and eliminates information loss caused by reliance on a single feature dimension.

The present disclosure maps the fused features to a Marke value range of 0-100 via a Softmax function, with a design compatible with existing pain assessment systems (e.g., VAS/NRS scales). The quantification results are independent of verbal feedback from subjects. Unlike common EEG analysis methods that rely on complex handcrafted feature engineering, the disclosed scheme enables end-to-end automated processing, eliminating subjective interpretation bias by providing an objective assessment tool for special populations such as aphasic patients and children.

According to a preferred embodiment, the processor further includes a Bayesian update module, which is configured to receive the objective pain quantification indicator and calculate a 95% confidence interval of the objective pain quantification indicator based on model cognitive uncertainty and observational noise level to generate the credibility of the assessment result of the 95% confidence interval. If the confidence interval width exceeds a predetermined threshold, the Bayesian update module dynamically adjusts observational noise parameters to optimize feature weight allocation. The Bayesian update module dynamically adjusts feature weights using a variational inference algorithm to quantify prediction uncertainty. This mechanism automatically attenuates the contribution of interfering frequency bands in response to device variability and individual physiological noise (e.g., EMG artifacts), significantly enhancing cross-device consistency and resistance to interference. Unlike common static models susceptible to hardware parameters or environmental noise, the disclosed scheme optimizes model parameters using real-time feedback to ensure consistent pain scoring performance across various scenarios.

According to a preferred embodiment, the frequency-band segmentation module is configured to extract time-frequency energy distribution features for each frequency band in the global time-frequency feature matrix based on a learnable frequency-domain filter, and to dynamically assign feature weights to the extracted time-frequency energy distribution features based on a cross-frequency-band attention mechanism, thereby fusing energy- and phase-coupling characteristics across the frequency bands to generate band-specific time-frequency feature matrices.

The frequency-band segmentation module, using learnable frequency-domain filters and a cross-frequency-band attention mechanism, significantly improves the dynamic representation of time-frequency energy distribution features. The innovation lies in overcoming the limitations of common fixed frequency-band segmentation by adaptively learning frequency-domain filter parameters to precisely capture differential neural oscillation patterns across frequency bands. An attention mechanism dynamically fused energy and phase coupling characteristics across frequency bands. This multi-band collaborative modeling technique achieves nonlinear decoupling of pain-related neural activity frequency-domain features, providing an interpretable quantitative basis for revealing the frequency-specific characteristics of pain physiology.

According to a preferred embodiment, the first assessment submodule is configured to extract the adjacency matrix by: based on a Gumbel-Softmax method, transforming the last time step features into a connectivity graph of the electrodes, thereby forming adjacency matrix representing the degree of neural activity coupling across cortical regions.

The Gumbel-Softmax-based graph generation network innovatively enables mathematical representation of dynamic functional connectivity topology. By transforming the last time step features into connectivity graphs with probabilistic meaning, this technique effectively eliminates loss of spatiotemporal coupling feature seen in common static brain network modeling. The adjacency matrix generated by the present disclosure not only quantifies the synchrony strength of neural activity between cortical regions but also preserves the temporal association of functional connectivity throughout dynamic evolution by means of differentiable properties.

According to a preferred embodiment, the second assessment submodule is configured to compute a class probability distribution of the fused feature vector by: calculating a class probability distribution of the fused feature vector; and based on a Softmax function, outputting normalized values in a range of [0, 1], and mapping the normalized values to an objective pain quantification indicator within a range of [0, 100] through linear transformation.

The Marke value normalization system, using the Softmax function, enables continuous probabilistic mapping of pain intensity through an end-to-end deep learning framework. This overcomes the discretization limitations of common classification models by constructing a smooth mapping relationship between neural features and quantification indicators using the multi-scale feature fusion capability of the second assessment submodule. Its probabilistic output ensures clinical interpretability of values within the 0-100 range and reduces the impact of inter-subject distribution heterogeneity through nonlinear normalization, providing a technical foundation for standardization of indicators in multicenter studies.

According to a preferred embodiment, the objective pain quantification indicator is a Marke value ranging from 0 to 100, in units of Marke; wherein the greater the Marke value, the more intense the pain indicated by the pain intensity mapping.

The standardized construction of the Marke value system marks a paradigm shift in pain assessment from subjective description to objective measurement. By rigorously defining the numerical range, units, and intensity mapping relationship, the present disclosure innovatively establishes a neuro-biomarker system with explicit clinical semantics. Its continuous value characteristics enable detection of sub-threshold variations in pain intensity, and the 100-level quantization gradient significantly outperforms the common 11-point system of the VAS scale, providing a high-sensitivity tool for dynamic pain monitoring and therapeutic efficacy assessment in medical applications requiring high precision.

In a second aspect, the present disclosure provides an assessment model for objective pain assessment wherein the assessment model is implemented by an assessment module, the assessment module comprising a first assessment submodule and a second assessment submodule. The first assessment submodule is configured to use a bidirectional gated recurrent unit to extract time-series features of pain-related EEG signals from band-specific time-frequency feature matrices; extract last time step features and an adjacency matrix from the time-series features; and based on the last time step features and the adjacency matrix, generate a first feature vector representing global association patterns among electrodes used for acquiring EEG signals. The second assessment submodule is configured to perform convolution on the time-series features to generate a second feature vector that contains local spatiotemporal dynamic features of the EEG signals; concatenate the first and second feature vectors along a feature dimension to form a fused feature vector containing both the global association patterns and the local spatiotemporal dynamic features; and calculate a class probability distribution of the fused feature vector, perform normalization, and generate an objective pain quantification indicator corresponding to the EEG signals. The time-series features of the pain-related EEG signals are extracted from a global time-frequency feature matrix, which is generated from the pain-related EEG signals through fast Fourier transform. The global time-frequency feature matrix is segmented by a frequency-band segmentation module in a frequency domain into band-specific time-frequency feature matrices of five frequency bands associated with pain perception, namely δ, θ, α, β, and γ.

According to a preferred embodiment, the first assessment submodule is configured to extract the adjacency matrix by: based on a Gumbel-Softmax method, transforming the last time step features into a connectivity graph of the electrodes, thereby forming adjacency matrix representing the degree of neural activity coupling across cortical regions.

According to a preferred embodiment, the second assessment submodule is configured to compute a class probability distribution of the fused feature vector by: calculating a class probability distribution of the fused feature vector, and based on a Softmax Function, outputting normalized values in a range of [0, 1], and mapping the normalized values to an objective pain quantification indicator within a range of [0, 100] through linear transformation.

According to a preferred embodiment, the objective pain quantification indicator is a Marke value ranging from 0 to 100, in units of Marke; wherein the greater the Marke value, the more intense the pain indicated by the pain intensity mapping.

### BRIEF DESCRIPTION OF DRAWINGS

FIG. 1 is a schematic diagram illustrating the module connection relationship in an objective pain assessment apparatus according to one embodiment of the present disclosure;

FIG. 2 is a schematic diagram illustrating the module connection relationship in an objective pain assessment apparatus according to another embodiment of the present disclosure;

FIG. 3 is an EEG of a first subject for the present disclosure;

FIG. 4 is an EEG of a second subject according to the present disclosure;

FIG. 5 is a schematic diagram of pain data for the first subject according to the present disclosure;

FIG. 6 is a schematic diagram of pain data for the second subject according to the present disclosure;

FIG. 7 is a schematic diagram of classification results of a confusion matrix according to the present disclosure; and

FIG. 8 is a regression scatter plot depicting the relationship between Marke values and actual pain scores according to the present disclosure;

FIG. 9 is a schematic diagram illustrating the module connection relationship in an objective pain assessment apparatus according to another embodiment of the present disclosure.

### DETAILED DESCRIPTION

Detailed description is provided below with reference to the accompanying drawings.

Pain, as the fifth vital sign, presents a fundamental clinical challenge—the lack of objective biomarkers. Current pain assessment methods mainly rely on subject self-reporting and scale-based assessment tools (e.g., VAS, NRS, DN4, and IDpain). The core issue lies in the subjective assessment model, where subjects' pain descriptions are influenced by subjective emotional states, cognitive factors, levels, and environmental influences conditions. This subjectivity leads to assessment results that may not accurately reflect clinical reality, such as scores that may be inflated due to subjective pain anxiety or inaccurate reporting from subjects with limited communication abilities. Additionally, differing assessments by various medical professionals for the same subject can lead to inconsistent treatment plans and therapeutic evaluations outcomes, with these discrepancies being particularly pronounced in complex pain management requiring multidisciplinary collaboration.

Common assessment methods have inherent shortcomings in continuous or dynamic monitoring. One-time or intermittent discrete time-point scale assessments fail to capture real-time fluctuations in pain intensity, such as daily variations in postoperative pain or sudden exacerbations of neuropathic pain. More importantly, simple numerical rating scales cannot adequately reflect the complex underlying mechanisms involved in chronic pain, such as central sensitization and neuroplasticity changes, resulting in a superficial representation of the pathological essence. These issues are further exacerbated in special populations, such as children, elderly subjects, and individuals with impaired consciousness, potentially leading to the dual risks of excessive analgesic overuse or insufficient undertreatment, both of which directly compromise patient clinical outcomes decision-making.

In research and healthcare pain management, the limitations of subjective assessment create significant bottlenecks. For instance, the lack of standardized assessment criteria in clinical trials can reduce the sensitivity of efficacy determination, increasing the risk of biased research conclusions. The absence of objective pain measurement indicators also hinders cross-institutional data comparison and the standardization of diagnostic and treatment protocols, thereby limiting the overall advancement of pain management.

To address the limitations of the existing art, the present disclosure aims to establish an objective pain intensity indicator based on EEG signals and assessment module 190. The integrated model within the assessment module 190 is collectively referred to as the digital twin pain brain model.

The core principle of the present disclosure is as follows:

The global time-frequency feature matrix of the EEG signals is segmented in a frequency domain into band-specific time-frequency feature matrices of five frequency bands associated with pain perception, namely δ, θ, α, β, and γ. Time-series features of the EEG signals are extracted from the band-specific time-frequency feature matrices, and last time step features and an adjacency matrix are extracted from the time-series features. A first feature vector representing global association patterns among electrodes used for acquiring EEG signals is generated based on the last time step features and the adjacency matrix. Through performing convolution on the time-series features, a second feature vector containing local spatiotemporal dynamic features of the EEG signals is generated, then the first and second feature vectors are concatenated along a feature dimension to form a fused feature vector containing both global association patterns and local spatiotemporal dynamic features. By computing a class probability distribution of the fused feature vector and performing normalization, an objective pain quantification indicator corresponding to the EEG signals is obtained. A Bayesian update module is used to receive the objective pain quantification indicator, and calculate a 95% confidence interval of the objective pain quantification indicator according to model cognitive uncertainty and observational noise level to generate the credibility of assessment result of the 95% confidence interval; wherein if the confidence interval width exceeds a predetermined threshold, the Bayesian update module dynamically adjusts observational noise parameters to optimize feature weight allocation. Preferably, the training data used for training the assessment module 190 in the present disclosure are pain-related EEG signals. These training data may be sourced from multiple databases, including the digital twin pain brain model (database), as well as the PAIN Consortium Database and the INCF Pain-EEG Database in the US. The digital twin pain brain model (database) is the first whole-brain-scale digital twin brain (DTB) model developed in China, capable of simulating dynamic brain functions and applied in studies such as epilepsy and steady-state visual evoked potentials (SSVEP). The digital twin pain brain model (database) provides pain-related EEG signals data. The PAIN Consortium Database includes multi-center EEG signal data from over 3,000 participants across 12 countries. The INCF Pain-EEG Database contains EEG signal data from approximately 150 to 200 participants in Europe and North America, including 100 acute pain patients and 50 chronic pain patients.

Preferably, the objective pain quantification indicators proposed by the present disclosure are Marke values. The Marke values are generated through the fusion of data extracted from multimodal bio-signals and reflect both physiological and psychological responses of subjects to pain. In the present disclosure, a processor 110 processes these bio-signals through a multi-task learning model and converts the analog pain response signals into quantification indicators with a numerical range of 0 to 100. These quantification indicators are referred to as Marke values. The larger the mapped Marke value, the stronger the pain, i.e., the higher the pain intensity (see Table 1).

The objective pain quantification indicators of the present disclosure are compatible with existing clinical scales, such as the VAS (0-10 points) and NRS (0-10 levels), and can be directly converted by dividing by 10. A difference represented by 1 (one) Marke corresponds to a distinguishable change in pain intensity. One Marke is defined as the range of neural signal fluctuations in healthy subjects in a resting state.

Based on the fundamental principle as described previously, the present disclosure may provide a method and apparatus for digital twin-based pain perception modeling. The present disclosure further provides a system and method for EEG-driven pain quantification and analysis. The present disclosure may further provide an objective pain measurement algorithm and an apparatus using the algorithm. The present disclosure may further provide a method and apparatus for generating an EEG-based pain assessment model. The present disclosure may further provide a method for visualization and digitalization of vital signs. The present disclosure may further provide a multidimensional pain assessment and display platform. The present disclosure may further provide a system and method for intelligent pain monitoring and analysis. The present disclosure may further provide a method for high-precision pain-related brain mapping modeling and real-time monitoring. Last but not least, the present disclosure may further provide a digital twin-based solution for individualized pain management.

The present disclosure may further provide a storage medium, which stores an encoded program for the assessment model to execute the assessment method of the present disclosure for objective pain assessment. The present disclosure further relates to a processor 110, which is configured to execute the assessment method of the present disclosure for objective pain assessment based on the assessment module 190 and output objective indicator information related to pain. The objective indicator information may be data information or analog signals, as long as the output enables the terminal to read and display the objective pain indicators.

### Embodiment 1

The present embodiment illustrates the method of the present disclosure by taking an assessment apparatus 100 for objective pain assessment as an example.

Preferably, the assessment apparatus 100 of the present disclosure is connected to an EEG acquisition device 200 in a wired or wireless manner. The assessment apparatus 100 is further connected to at least one terminal 300 in a wired or wireless manner, so that the terminal 300 receives objective pain quantification indicators that represent pain levels, information related thereto, and information about how the assessment apparatus processes EEG signals.

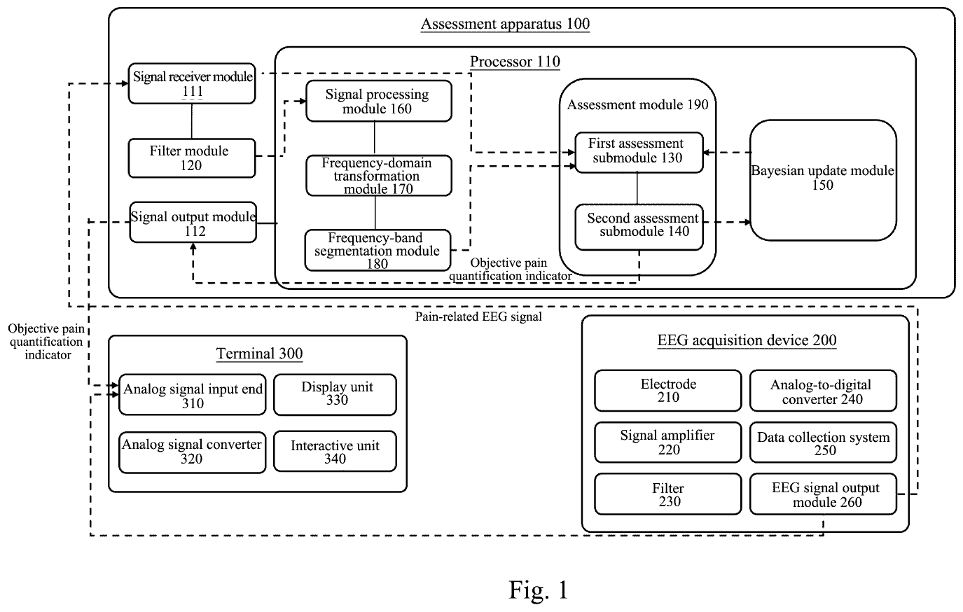

Specifically, as shown in FIG. 1, the assessment apparatus 100 may include a processor 110. The processor 110 may be implemented as a GPU, a CPU, or similar components. The processor 110 may further be equipped with a memory. The memory is configured to store data received, processed, and ultimately generated by the processor 110. Preferably, the processor 110 includes hardware modules and combinations thereof.

Preferably, as shown in FIG. 1, the assessment apparatus 100 may include the processor 110 and a filter module 120. In particular, the filter module 120 is pre-installed upstream of the processor 110 along the data stream to preprocess the received EEG signals by performing noise cancellation and filtering. Preferably, the filter module 120 may be integrated in the processor 110 as a part of the processor 110. Preferably, as shown in FIG. 1, the processor 110 may further include a Bayesian update module 150, a signal processing module 160, a frequency-domain transformation module 170, a frequency-band segmentation module 180, and an assessment module 190.

Preferably, the signal processing module 160 is, for example, composed of an analog-to-digital converter (ADC) and a digital signal pre-processing unit, for realizing signal sampling and digitization. The frequency-domain transformation module 170 may be a digital signal processor (DSP) or a field-programmable gate array (FPGA), specifically configured to perform mathematical operations such as fast Fourier transform (FFT). The frequency-band segmentation module 180 may be a filter set or a multi-channel frequency division circuit, for the frequency band segmentation function. The assessment module 190 may be an embedded microprocessor or a neural network accelerator chip, on which the assessment model, including the bidirectional gated recurrent unit (BiGRU) 132 and the Gumbel-Sampler unit 133, is deployed. In other words, the assessment model is operated by the assessment module 190.

Preferably, the signal processing module 160 transmits the resampled EEG signals to the frequency-domain transformation module 170 through a high-speed parallel bus, with this connection ensuring the integrity and real-time transmission of the sampled data. A bidirectional data channel is implemented between the frequency-domain transformation module 170 and the frequency-band segmentation module 180, transmitting the global time-frequency feature matrix of the EEG signals generated via Fourier transform while retaining the transmission path of the raw time-series signals. The frequency-band segmentation module 180 partitions the received EEG time-series features into five frequency bands via a multiplexed interface, and distributes the corresponding band-specific time-frequency feature matrices to the assessment module 190. This star topology supports parallelized frequency-band feature analysis. The timing synchronization among the modules is centrally coordinated by the internal clock tree of the processor 110, and data communication is achieved through a shared memory or direct memory access (DMA) mechanism, ensuring the continuity of the system processing pipeline. In the case of an integrated design, the assessment module 190 may be packaged within a system-on-chip (SoC) and interconnected via a network-on-chip (NoC), while external expansion modules are physically connected via standard communication interfaces.

As shown in FIG. 1, the assessment apparatus 100 further comprises a signal receiver module 111 and a signal output module 112. The signal receiver module 111 is used to receive EEG signals. The signal output module 112 is used to output the generated objective indicators that represent pain levels and information related thereto, and may also output information about how the assessment apparatus 100 processes EEG signals.

As shown in FIG. 1, the EEG acquisition device 200 is used to acquire EEG signals from subjects. The EEG acquisition device 200 includes electrodes 210, a signal amplifier 220, a filter 230, an analog-to-digital converter (ADC) 240, a data collection system 250 and an EEG signal output module 260. The electrodes 210 may be connected to the signal amplifier 220 in a wired or wireless manner to receive electrode signals from subjects. The signal amplifier 220 is used to amplify the EEG signals, and the filter 230 is used to remove noise and interference. The analog-to-digital converter 240 converts analog signals into digital signal for further processing. The EEG signal output module 260 is connected to the signal receiver module 111 of the assessment apparatus 100, thereby transmitting the EEG signals generated by the EEG acquisition device 200 to the assessment apparatus 100 for Fourier transform.

As shown in FIG. 1, the terminal 300 may be a mobile, portable electronic device, such as a smartphone or a tablet computer, or an immovable computer terminal, such as a desktop computer or a workstation. The terminal 300 includes one or more of an analog signal input end 310, an analog signal converter 320, a display unit 330, an interactive unit 340, and a processor unit.

The EEG signals output by the EEG acquisition device 200 may further be input to the analog signal input end 310 of the terminal 300, for example, through a 3.5 mm audio jack or a dedicated interface. The analog signal converter 320 is used to convert these analog signals into digital signals, ensuring the signals can be processed by a computer. The analog signal converter 320 may be, for example, a built-in analog-to-digital converter or an external audio interface device. The display unit 330 is used to display the processed EEG signals and their processing process in real-time, which may be a high-resolution LCD screen or a touch display, allowing users to visually observe and analyze the signals intuitively. The interactive unit 340 includes input devices, such as a keyboard, a mouse, or a touch screen, which allow users to interact with the terminal 300 through clicking, sliding, or entering commands. The processor unit may be, for example, a central processing unit (CPU) or a graphics processing unit (GPU) integrated in the terminal 300, used to execute complex signal analysis and data processing tasks. The analog signal input end 310 of the terminal 300 is connected to the signal output module 112 of the assessment apparatus 100 in a wired or wireless manner, ensuring that scores of the objective pain indicators can be displayed and monitored.

As shown in FIG. 1, the EEG acquisition device 200 and the assessment apparatus 100 are connected through the signal receiver module 111, so that the collected EEG signals can be sent to the assessment apparatus 100 through the signal receiver module 111. The signal receiver module 111 is connected to the filter module 120. The filter module 120 receives the EEG signals from the EEG acquisition device 200 through the signal receiver module 111 for secondary filtration. The filter module 120 then sends the post-filtration EEG signals to the signal processing module 160. After receiving the post-filtration EEG signals from the filter module 120, the signal processing module 160 resamples the EEG signals and sends the resampled EEG signals to the frequency-domain transformation module 170. After receiving the resampled EEG signals from the signal processing module 160, the frequency-domain transformation module 170 obtains the global time-frequency feature matrix through Fourier transform and sends the global time-frequency feature matrix to the frequency-band segmentation module 180. After receiving the global time-frequency feature matrix from the frequency-domain transformation module 170, the frequency-band segmentation module 180 segments the global time-frequency feature matrix in a frequency domain into band-specific time-frequency feature matrices corresponding to five frequency bands (δ, θ, α, β, and γ) related to pain perception. The frequency-band segmentation module 180 sends the band-specific time-frequency feature matrices corresponding to the five frequency bands (δ, θ, α, β, and γ) to the assessment module 190. After receiving the band-specific time-frequency feature matrices corresponding to the five frequency bands (δ, θ, α, β, and γ) from the frequency-band segmentation module 180, the assessment module 190 performs the computation of the objective pain quantification indicators.

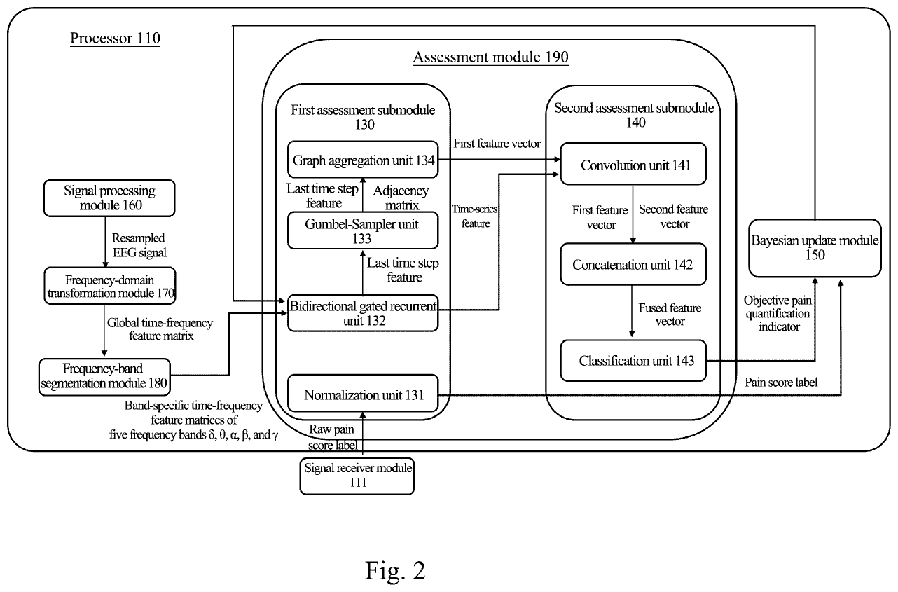

Preferably, as shown in FIG. 2, the first assessment submodule 130 in the assessment module 190 includes a normalization unit 131, a bidirectional gated recurrent unit 132, a Gumbel-Sampler unit 133, and a graph aggregation unit 134. After receiving raw pain score labels from the signal receiver module 111, the normalization unit 131 performs normalization to obtain the normalized pain score labels. The normalization unit 131 sends the normalized pain score labels to the Bayesian update module 150.

After receiving the band-specific time-frequency feature matrices corresponding to the five frequency bands (δ, θ, α, β, and γ) from the frequency-band segmentation module 180, the bidirectional gated recurrent unit 132 processes them and outputs time-series features and last time step features. The bidirectional gated recurrent unit 132 sends the last time step feature to the Gumbel-Sampler unit 133. The bidirectional gated recurrent unit 132 sends the time-series features to a convolution unit 141 in the second assessment submodule 140.

After receiving the last time step features from the bidirectional gated recurrent unit 132, the Gumbel-Sampler unit 133 computes an adjacency matrix. The Gumbel-Sampler unit 133 sends the last time step features and the adjacency matrix to the graph aggregation unit 134. After receiving the last time step features and the adjacency matrix from the Gumbel-Sampler unit 133, the graph aggregation unit 134 computes first feature vector and then sends it to the convolution unit 141 in the second assessment submodule 140. After receiving the time-series features from the bidirectional gated recurrent unit 132, the convolution unit 141 computes second feature vector and then sends both the first and second feature vectors to a concatenation unit 142 to compute a fused feature vector, which will be sent to a classification unit 143. After receiving the fused feature vector from the concatenation unit 142, the classification unit 143 computes the objective pain quantification indicators and sends them to the Bayesian update module 150.

After receiving the normalized pain score labels from the normalization unit 131, the Bayesian update module 150 constructs a variational loss function. Based on this loss function, the Bayesian update module 150 updates the weight parameters of the bidirectional gated recurrent unit 132 using a reparameterization gradient algorithm. After receiving the objective pain quantification indicators from the classification unit 143, the Bayesian update module 150 performs confidence interval calculation, integrating model cognitive uncertainty and observational noise levels to generate the credibility of the 95% confidence interval assessment results; it constructs a threshold-triggering mechanism, automatically adjusting the observational noise parameters and recalibrating the allocation ratio of feature weights when the confidence interval width exceeds a preset threshold; it further suppresses outliers and initiates a parameter retraining process for predictions that continuously deviate from labeled values, thereby reducing interference from device noise and physiological artifacts.

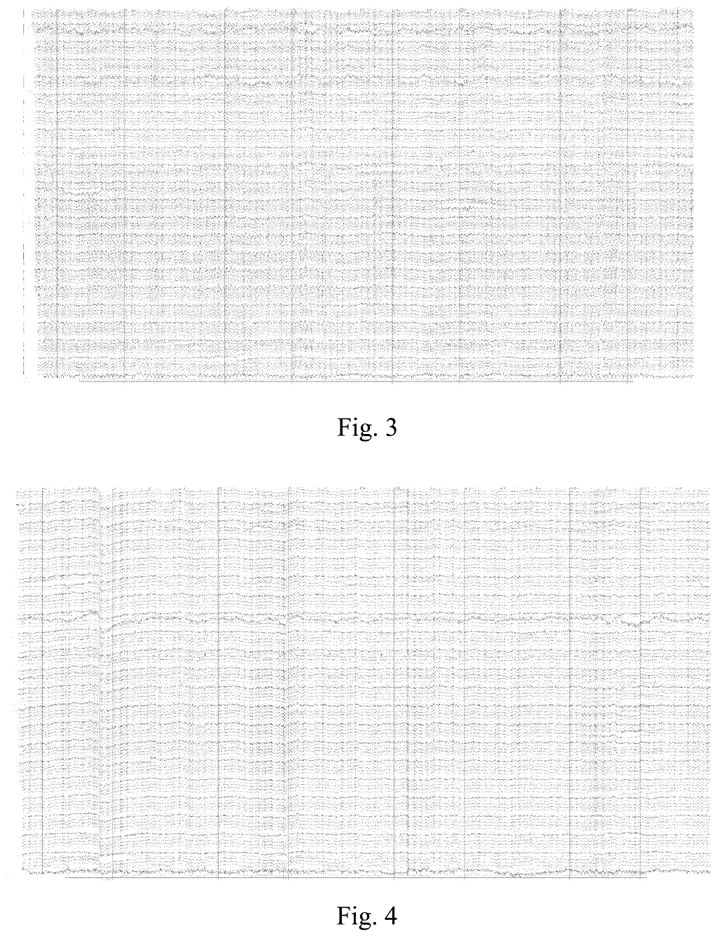

Before the assessment apparatus 100 begins assessment, EEG signals from a subject need to be collected using the EEG acquisition device 200 (see FIG. 3 and FIG. 4).

Preferably, a high-precision EEG acquisition device 200 is used, with appropriate gain and filter parameters set to ensure the clarity and accuracy of the signals. Electrodes 210 suitable for the subject are selected. When a target zone is determined on the subject's head, the electrodes 210 are affixed to the target zone, ensuring that the multiple electrodes 210 make direct contact with the target zone on the subject's head. Preferably, conductive gel is used to ensure good contact between the electrodes 210 and the scalp in the target zone.

The electrodes 210 transmits raw EEG signals to the signal amplifier 220 in a wired or wireless manner. The signal amplifier 220, through impedance matching and low-noise amplification circuits, amplifies microvolt-level EEG signals to a processable range while suppressing common-mode interference. The amplified signals are then preprocessed by the filter 230, where a high-pass filter (with a cutoff frequency of 0.5 Hz) eliminates baseline drift, and a low-pass filter (with a cutoff frequency of 75 Hz) removes electromyographic (EMG) noise, thereby forming analog waveforms consistent with bioelectrical signal characteristics. The analog-to-digital converter (ADC) 240 digitalizes the filtered signals at a sampling rate no lower than 250 Hz, with its 24-bit resolution ensuring precise capture of subtle potential changes. The data acquisition system 250 performs temporal alignment and buffering of multi-channel digital signals, and the EEG signal output module 260 encapsulates the synchronized EEG signal data stream into data packets compliant with the IEEE 11073 protocol.

These data packets are transmitted via a single-mode optical fiber cable to the signal receiver module 111 of the assessment apparatus 100 and then forwarded to the memory by the signal receiver module 111. Preferably, the optical fiber interface employs an SFP+ optical module to achieve high-speed transmission at 10 Gbps, with its anti-electromagnetic interference properties preventing degradation of the signal-to-noise ratio during transmission. Additionally, the signal receiver module 111 of the assessment apparatus 100 verifies data integrity using a cyclic redundancy check (CRC) mechanism.

The assessment apparatus 100 of the present disclosure is configured to perform the following operations:

It preprocesses received EEG signals through steps including secondary filtering and resampling.

When the filter module 120 is integrated within the assessment apparatus 100, the filter module 120 receives the EEG signals via the signal receiver module 111 and performs secondary filtering to achieve noise reduction. Alternatively, the filter module 120 may also be positioned external to the assessment apparatus 100, directly connected to the EEG signal output module 260 of the EEG acquisition device 200 to receive the EEG signals. This embodiment primarily describes the exemplary configuration where the filter module 120 is integrated within the assessment apparatus 100.

Insufficient filtering may result in the following defects:

Although the EEG signals output by the EEG acquisition device 200 have undergone preliminary noise reduction, their fixed cutoff frequency range (0.5-75 Hz) cannot fully suppress high-frequency residual noise in digital signals (e.g., EMG harmonic interference) or the sidelobe energy of power-line interference. Without secondary filtering, high-frequency noise components (>75 Hz) in EEG signals may leak into the β and/or γ frequency bands (12-100 Hz) during subsequent frequency band segmentation due to spectral leakage, leading to distorted power calculation in the target frequency bands. Moreover, unfiltered transient artifacts (e.g., eye blinks or motion artifacts), which manifest as non-stationary spike signals in the time domain, may be misinterpreted as abnormal functional connectivity between brain regions after feature extraction by the first assessment submodule 130 in the assessment apparatus 100. For example, transient high-power values caused by eye blink artifacts at frontal electrodes may be erroneously interpreted by the first assessment submodule 130 as false features indicating enhanced connectivity in the fronto-limbic network.

Residual power-line noise (50 Hz and its harmonics) manifests as common-mode interference across all scalp electrodes in the spatial dimension, which can impair the second assessment submodule 140's ability to distinguish region-specific neural activities. Unfiltered common-mode noise may mask true pain-related spatial features (e.g., increased θ-band power in the ACC), causing the second assessment submodule 140 to allocate weights away from critical brain regions. Additionally, unfiltered signal variations (e.g., device noise or environmental electromagnetic interference) introduce random fluctuations unrelated to pain, increasing the risk of overfitting in the first assessment submodule 130. The first assessment submodule 130 may mistake noise patterns for pain-specific features, resulting in reduced generalization across datasets.

To address these issues with insufficient filtering, the present disclosure employs the filter module 120 to perform secondary filtering on EEG signals, further suppressing residual interference before frequency-domain analysis, thereby preventing noise components from affecting the accuracy of spatiotemporal feature extraction by the first assessment submodule 130 of the assessment apparatus 100.

Preferably, the filter module 120 includes a high-pass filter and a low-pass filter. The high-pass filter is connected to the signal receiver module 111. The high-pass filter and the low-pass filter are connected via a signal line.

The high-pass filter (with a cutoff frequency of 0.5 Hz) removes low-frequency noise from the received EEG signals, such as electrocardiographic interference and motion artifacts. The high-pass filter sends the EEG signals with low-frequency noise removed to the low-pass filter. Preferably, the low-pass filter (with a cutoff frequency of 75 Hz) within the filter module 120 removes high-frequency noise from the EEG signals, such as EMG interference and power-line noise, reducing low-frequency drift and high-frequency noise components. Preferably, the low-pass filter is connected to the signal processing module 160 to transmit the secondary-filtered EEG signals to the signal processing module 160 within the assessment apparatus 100.

After receiving the filtered EEG signals from the low-pass filter in the filter module 120, the signal processing module 160 resamples the EEG signals.

The EEG signals after the secondary filtering are time-domain signals, with a sampling rate of 500 Hz at this time. Without resampling, the length of their time series doubles. For a 10-minute EEG signal recording process, the number of channels of electrode 210 is 128 leads, and the data volume will increase from approximately 1.08 GB (250 Hz) to 2.16 GB (500 Hz). The time consumption of the first assessment submodule 130 for extracting spectral features scales quadratically with the data volume to be processed, resulting in a noticeable increase in required computation time.

Most pain-related features are concentrated within the five frequency bands, namely δ, θ, α, β, and γ, and thus a sampling rate of 250 Hz satisfies the Nyquist criterion (supporting signals up to 125 Hz). High-sampling-rate data contain more individual-specific noise details (e.g., micro-EMG tremor), which may be mistakenly associated with pain-related features by the subsequent second assessment submodule 140 in the present disclosure. From the perspective of data transmission, the memory bandwidth of the assessment apparatus 100 (approximately 25.6 GB/s) reaches a utilization rate of up to 93% under a 500 Hz data stream, potentially leading to data transmission bottlenecks and causing real-time processing delays that exceed the clinically tolerable threshold (>200 ms).

To address this, the present disclosure provides a signal processing module 160 in the processor 110 to resample the EEG signals, reducing the original sampling rate from 500 Hz to 250 Hz, thereby compressing the data volume while preserving information on the frequency bands associated with pain perception, achieving an optimal balance between accuracy and efficiency.

The signal processing module 160 includes an integrated analog-to-digital converter (ADC) and a digital signal pre-processing unit. The ADC continuously digitalizes the EEG signals at a sampling rate of 500 Hz, after which the digital signal pre-processing unit performs anti-aliasing filtering using a low-pass filter (with a cutoff frequency of 125 Hz) to remove high-frequency aliasing interference. Subsequently, downsampling at an integral multiple of 250 Hz is achieved through dot-interlaced sampling. This sampling rate fully covers the five frequency bands associated with pain perception, namely δ, θ, α, β, and γ. The signal processing module 160 sends the resampled EEG signals to the frequency-domain transformation module 170.

This downsampling process reduces the data volume by 50%, while achieving the following three core optimizations: first, it improves the signal-to-noise ratio in the target frequency bands by filtering out non-physiological signals above 125 Hz, such as EMG interference harmonics; second, it enhances the model's generalization capability by standardizing the sampling rate, eliminating differences in acquisition parameters across devices; third, it reduces interference in subsequent time-frequency analysis and functional connectivity modeling by suppressing high-frequency transient noise, such as motion artifacts. This design achieves an optimal balance between computational efficiency and feature fidelity while preserving pain biomarkers, such as β/γ oscillation phase synchrony.

After receiving the resampled EEG signals from the signal processing module 160, the frequency-domain transformation module 170 obtains a global time-frequency feature matrix using Fourier transform, as shown in FIG. 2. The EEG signals received from the signal processing module 160 are time-domain signals X∈ [symbol] ^{B×N×T}, where B denotes the batch size, N denotes the number of electrodes, and T denotes the time-series length. The data of the time-domain signal X∈ [symbol] ^{B×N×T} can be represented as a tensor with dimensions of {48, 128, 38}, where 48 denotes the frequency component, 128 denotes the number of electrodes, and 38 denotes the time-series length.

The frequency-domain transformation module 170 segments the time-series length T into K overlapped time windows, each with a length L and a sliding step size of H, where T=L+(K−1) H.

After windowing, the signal dimension becomes X_{Win}∈ [symbol] ^{B×D×N×K}.

The frequency-domain transformation module 170 applies a Hanning window to each time window to suppress spectral leakage:

$$ω ( l ) = 0.5 * [ 1 - cos ( \frac{2 π l}{L - 1} ) ] , l = 0 , 1 , … , L - 1 .$$

The windowed signal is:

$${X}_{W i n}^{W i n} [ b , n , k , l ] = {X}_{W i n} [ b , n , k , l ] \cdot ω ( l ) .$$

The frequency-domain transformation module 170 performs discrete Fourier transform (DFT) on each time window, as expressed by the equation below:

$${X}_{STFT} [ b , n , k , l ] = {\sum }_{l = 0}^{L - 1} {X}_{W i n}^{W i n} [ b , n , k , l ] \cdot {e}^{- 2 π j d l / L} .$$

The frequency-domain transformation module 170 outputs a complex time-frequency matrix X_{STFT}∈ [symbol] ^{B×D×N×K} where D=[L/2]+1 is the number of single-sided spectral components.

The frequency-domain transformation module 170 computes the logarithmic power spectrum of the complex time-frequency matrix and takes the results as a global time-frequency feature matrix:

$${X}_{T F} [ b , n , k , d ] = 10 \cdot {log}_{10} ( {\vert {X}_{S T F T} [ b , n , k , d ] \vert}^{2} + ε ) .$$

Ultimately, the frequency-domain transformation module 170 outputs a real-valued time-frequency feature tensor, or a global time-frequency feature matrix X_{TF}∈ [symbol] ^{B×D×N×K} where ε=1e−6.

Through the aforementioned generation process of the global time-frequency feature matrix (segmenting→windowing→STFT→logarithmic power spectrum), the present disclosure achieves quantitative representation of the dynamic time-frequency characteristics of the EEG signals. First, the frequency bands, δ (1-4 Hz), θ (4-9 Hz), α (9-12 Hz), β (12-25 Hz), and γ (25-100 Hz) of human EEG signals correspond to different neural activity patterns. For example, acute pain often induces increased power in the β/γ frequency bands, whereas chronic pain is associated with sustained oscillations in the θ frequency band. Through frequency-channel partitioning, the global time-frequency feature matrix can accurately isolate power density variations across these frequency bands. Secondary, pain perception exhibits temporal evolution characteristics, including latency phase (characterized by increased low-frequency power)→peak phase (marked by transient high-frequency bursts)→fading phase (featuring cross-frequency synchronization). The window dimension K of the global time-frequency feature matrix corresponds to the temporal evolution axis (i.e., the step size covered by each window), enabling the localization of time-frequency features to specific time windows and frequency bands. By analyzing power trajectories along the dimension K (such as integrating power in the γ frequency band across windows k=50-80), the dynamic response pattern to pain stimulation can be quantified.

After performing Fourier transform, the frequency-domain transformation module 170 standardizes the global time-frequency feature matrix.

The equation for this purpose is:

$${X}_{n o r m} = \frac{{X}_{TF} - {μ}_{X}}{{σ}_{X}} .$$

In the above equation, μ_{X} denotes the mean matrix across all dimensions, and σ_{X} denotes the standard deviation matrix.

The application of discrete Fourier transform in the above steps provides the following advantages for the subsequent identification of pain intensity biomarkers:

First, it reveals the neural encoding mechanisms of pain. From the post-DFT EEG signals, frequency-band energy features closely related to pain can be extracted. These frequency-band energy values are then converted into Marke objective pain indicators ranging from 0 to 100 through nonlinear mapping, thereby, for the first time, assigning standardized biomarkers to the subjective perception of “pain”.

Second, it enables multi-dimensional feature fusion analysis. The DFT output includes a three-dimensional feature matrix with 48 frequency components, 128 electrode channels, and 38 time windows, which not only reflects the spatial specificity of the prefrontal cortex and other brain regions involved in pain modulation but also captures transient γ-band burst patterns associated with acute pain episodes. Such integration of spatio-temporal-spectral features lays the foundation for constructing a digital twin pain brain model, thereby enabling the differentiation of pain subtypes such as neuropathic and inflammatory pain.

Third, it facilitates precision-oriented clinical decision-making. Through the DFT algorithm, the assessment apparatus 100 updates the pain assessment results every 2 to 5 seconds, realizing dynamic monitoring of postoperative pain. As demonstrated in clinical studies, a 30% reduction in γ-band power before and after treatment is significantly correlated with analgesic efficacy (R²=0.922, see FIG. 8). This objective efficacy assessment substantially reduces the risk of misjudgment caused by variability in subject self-reporting inherent in common practice.

The frequency-domain transformation module 170 then sends the standardized global time-frequency feature matrix to the frequency-band segmentation module 180, as shown in FIG. 2. After receiving the global time-frequency feature matrix from the frequency-domain transformation module 170, the frequency-band segmentation module 180 segments the global time-frequency feature matrix in a frequency domain into matrices corresponding to the five frequency bands associated with pain perception, namely δ, θ, α, β, and γ.

Specifically, the frequency-band segmentation module 180 partitions the standardized time-frequency feature matrix by frequency into matrices corresponding to the frequency bands δ (0-4 Hz), θ (4-9 Hz), α (9-12 Hz), β (12-25 Hz), and γ (25-100 Hz) based on the energy distribution across different frequency intervals, using a set of parallel FIR filters. For example, if the power spectral density estimate within a specific frequency interval indicates concentrated energy and this interval falls within the β frequency band, then the corresponding signal segment is assigned to the 0 band.

Preferably, the frequency-band segmentation module 180 extracts time-frequency energy distribution features from the band-specific time-frequency feature matrices using learnable frequency-domain filters and dynamically assigns weights to these features based on a cross-frequency-band attention mechanism, integrating energy and phase-coupling characteristics across frequency bands to generate time-frequency feature matrices with frequency-band specificity.

The frequency-band segmentation module 180, through learnable frequency-domain filters and a cross-frequency-band attention mechanism, significantly enhances the dynamic representation capability of time-frequency energy distribution features. Its innovation lies in overcoming the limitations of common fixed frequency-band segmentation by adaptively learning frequency-domain filter parameters to accurately capture the distinct neural oscillation patterns across different frequency bands, while employing an attention mechanism to dynamically integrate cross-band energy and phase-coupling characteristics. This multi-band collaborative modeling technique unprecedentedly achieves nonlinear decoupling of pain-related neural activity frequency-domain features, providing an interpretable and quantitative basis for revealing the frequency-band specificity underlying pain physiology mechanisms.

The frequency-band segmentation module 180 then sends the band-specific time-frequency feature matrices associated with the five frequency bands (δ, θ, α, β, and γ) to the assessment module 190, as shown in FIG. 2. With a communication connection established between the processor 110 and an external database, the band-specific time-frequency feature matrices for the frequency bands may be stored in the database. Preferably, the database may be a digital twin brain database connected to a third party.

From a neurological perspective, the physiological basis of the existing tripartite classification (θ: 4-8 Hz, α: 8-13 Hz, β: 13-30 Hz) is rooted in the neural oscillation mechanism supported by classical studies: α rhythms are associated with resting-state cortical synchronization, while β rhythms show clear associations with motor cortex activation and cognitive loads. This classification scheme has become paradigm-dependent in the field of neuro-electro-physiological research. Its advantage lies in the correspondence between frequency-band boundaries and interpretable transitions in neural activity patterns, including synaptic transmission efficiency and ion channel dynamics. The core challenge in transitioning to a five-band segmentation lies in the continuity of neural oscillatory mechanisms. For example, while pain-incurred activities in the γ band (30-100 Hz) are related to local neural cluster synchronization, the energy decays rapidly and is highly susceptible to contamination from EMG artifacts, making it difficult for common Fourier transform methods with fixed window lengths to achieve effective extraction. By contrast, the frequency-domain transformation module 170 of the present disclosure combines STFT and the use of Hanning windows to improve its capability of capturing high-frequency neural oscillations in three dimensions detailed below: The first is optimized time-frequency resolution. By setting the windowing parameters (i.e., window length L and the step length H), the temporal resolution in the γ band is enhanced to the millisecond scale (with the theoretical minimal temporal unit ΔT=1/ƒs) while maintaining sufficient frequency resolution (Δƒ=1/L). This parameter configuration enables effective separation of γ sub-bands (e.g., low γ: 25-50 Hz and high γ: 50-100 Hz) within the 25-100 Hz range, overcoming the issue of blurred high-frequency transient features caused by common FFT-based global spectral analysis. The second is enhanced artifact suppression. The use of the Hanning window function significantly improves spectral sidelobe attenuation, offering a reduction of over ten to several tens of decibels compared to the rectangular window, effectively reducing harmonic interference from EMG artifacts (primarily distributed within the 60-200 Hz range) in the γ frequency band. The last is preservation of dynamic features. By applying nonlinear compression through log-power spectral transformation (X_{TF} [b, n, k, d]), the dynamic range of low-amplitude γ oscillations is expanded. When γ power exhibits short bursts on the order of several tens of milliseconds in response to pain stimulation, the transformation enhances the sensitivity of weak-signal detection by more than 20%, for example, enabling effective capture of phase-amplitude coupling (PAC) features in the γ band between the prefrontal cortex and the ACC.

As shown in FIG. 1 and FIG. 2, after receiving the band-specific time-frequency feature matrices for the five frequency bands (δ, θ, α, β, and γ) from the frequency-band segmentation module 180, the assessment module 190 computes objective pain quantification indicators.

The assessment module 190 comprises a first assessment submodule 130 and a second assessment submodule 140. The first assessment submodule 130 is constructed from at least one graph generation network and serves to generate a first feature vector the represents the global association pattern among the electrodes 210 used to collect the EEG signals. The second assessment submodule 140 is constructed from at least one convolutional neural network and serves to generate objective pain quantification indicators corresponding to the EEG signals according to the first feature vector and a second feature vector generated by itself.

The first assessment submodule 130 is provided with a normalization unit 131, a bidirectional gated recurrent unit (BiGRU) 132, a Gumbel-Sampler unit 133, and a graph aggregation unit 134. The normalization unit 131 receives raw pain score labels from the signal receiver module 111 and performs normalization on the raw pain score labels. The BiGRU 132 is used to extract time-series features from the standardized band-specific time-frequency feature matrices of five frequency bands. The Gumbel-Sampler unit 133 is used to execute the Gumbel-Softmax method. The graph aggregation unit 134 is used to generate a first feature vector the represents the global association pattern among the electrodes 210 used to collect the EEG signals.

Specifically, the normalization unit 131 performs normalization on the received raw pain score labels.

The range of the raw pain score labels is: y_{reg}∈[0,10].

The normalization performed by the normalization unit 131 follows the equation:

$${y}_{n o r m} = \frac{{y}_{r e g}}{10} .$$

The range of the normalized pain score labels after normalization by the normalization unit 131 is: yₙₒᵣₘ∈[0,1],
where yₘᵢₙ=0, denoting the minimal pain score, and yₘₐₓ=10, denoting the maximal pain score.

In the regression task, the normalization unit 131 linearly maps the raw pain score labels to the range of [0, 1] through normalization. The linear normalization mapping of the raw pain score labels is consistent with the multi-dimensional neural coding mechanisms of pain. The normalization unit 131 sends the normalized pain score labels to the Bayesian update module 150 for loss computation.

Preferably, after receiving the standardized band-specific time-frequency feature matrices of five frequency bands (Xₙₒᵣₘ∈ [symbol] ^{B×D×N×K}) from the frequency-band [symbol] module 180, the BiGRU 132 processes the matrices and outputs time-series features: H_{BiGRU}=BiGRU(Xₙₒᵣₘ)∈ [symbol] ^{B×2D′×N}, where B denotes the batch size, N denotes the number of EEG electrodes (i.e., the number of channels), T denotes the time-series length, and D′ denotes the number of neurons in the GRU hidden layer. The dimension T is absorbed into the input dimension during the processing of the BiGRU 132 and does not appear in the output information. The bidirectional gated recurrent unit 132 sends the time-series features to the convolution unit 141 in the second assessment submodule 140.

The BiGRU 132 uses a time-series modeling mechanism to establish a multi-scale mapping between time-series features and pain intensity through the coordinated operation of reset gate and update gate. That is, the BiGRU 132 extracts the time-series features H_{BiGRU} along the temporal dimension to obtain the last time step features and the adjacency matrix from the extracted time-series features.

To this end, the BiGRU 132 captures the last time step (or terminal time step) features

$${H}_{B i G R U}^{( last )} ∈ {ℝ}^{B \times 2 D ′ \times N}$$

from the time-series features H_{BiGRU}. Due to the temporal characteristics of pain-related neural responses, the last time-step feature integrates the signal changes across all stages from the onset to the end of the stimulus.

Preferably, the hidden layer dimension of the BiGRU 132 is configured as D′=256. The BiGRU 132 processes the input by traversing both forward and backward temporal windows, concatenating the hidden states from each direction to form a 512-dimensional representation for each time window (2D′=2×256), thereby outputting a complete temporal feature. H_{BiGRU}=BiGRU(Xₙₒᵣₘ)∈ [symbol] ^{B×512×N}. The last time step feature is:

$${H}_{B i G R U}^{( last )}$$

∈ [symbol] ^{B×512×N}.

The BiGRU 132 then sends the last time step features to the Gumbel-Sampler unit 133, as shown in FIG. 2. After receiving the last time step features from the BiGRU 132, the Gumbel-Sampler unit 133 captures the cumulative dynamic patterns of the signal sequence based on the last time step feature

$${H}_{B i G R U}^{( last )} .$$

Due to the temporal cumulative nature of pain-induced neural responses (e.g., γ-band oscillations), the first assessment submodule 130 integrates dynamic changes across the entire time window by extracting the last time step features

$${H}_{B i G R U}^{( last )} .$$

For example the temporal integral of γ power in patients with postoperative pain shows a significantly higher correlation with Marke values than instantaneous measurements. This cumulative nature better aligns with the long-term potentiation (LTP) mechanism underlying central sensitization of pain.

The Gumbel-Sampler unit 133 converts the last time step feature

$${H}_{B i G R U}^{( last )}$$

into a connectivity graph between the electrodes 210 using the Gumbel-Softmax method, thereby forming an adjacency matrix that represents the degree of neural activity coupling between cortical regions. The equation for the connectivity graph (i.e., the adjacency matrix) is:

$$A = Gumbel - Sampling ( {H}_{G R U}^{( last )} ) ∈ {ℝ}^{N \times N} .$$

Its adjacency matrix element A_{i,j} denotes the association strength between the electrodes i and j, reflecting the functional connectivity patterns among brain regions.

Specifically, the adjacency matrix among the electrodes 210 is a square matrix, where each element represents the likelihood of a connection formed between the corresponding electrode pair. Assuming a network composed of multiple electrodes 210, there exists a probability of connection (i.e., the association strength) between each pair of the electrodes 210. The adjacency matrix element A_{i,j} of the adjacency matrix denotes the association strength between the electrodes i and j. A diagonal element A_{i,j} is usually 0 because an electrode 210 is not connected to itself. For the adjacency matrix A, if the association strength (i.e., the probability of connection) is symmetric, then A_{i,j}=A_{i,i}.

Assuming that there are 3 electrodes, the adjacency matrix A may be expressed as:

$$( \begin{matrix}0 & 0.5 & 0.2 \\ 0.5 & 0 & 0.3 \\ 0.2 & 0.3 & 0\end{matrix} ) ,$$

where the association strength is 0.5 between the first electrode and the second electrode, 0.2 between the first electrode and the third electrode, and 0.3 between the second electrode and the third electrode. The adjacency matrix A enables quantification and analysis of connections between the electrodes 210 or nodes.

In the case of 128-channel electrodes 210, let the set of electrodes be denoted as

$$𝒱 = {\{ {υ}_{i} \}}_{i = 1}^{128} ,$$

and the set of spatial coordinates as

$${\{ ( {x}_{i} , {y}_{i} , {z}_{i} ) \}}_{i = 1}^{128} .$$

Then the adjacency matrix A∈ [symbol] ^{128×128} can be formulated as:

$${A}_{i , j} = \{ \begin{matrix}0 & if i = j \\ {f}_{θ} ( {d}_{ij} ) \cdot {ρ}_{ij} & otherwise\end{matrix} ,$$

where the spatial decay term is:

$${f}_{θ} ( {d}_{i j} ) = exp ( - \frac{{d}_{ij}^{2}}{2 {σ}^{2}} ) ,$$

wherein dᵢⱼ=∥(xᵢ, yᵢ, zᵢ)−(xⱼ, yⱼ, zⱼ)∥₂ denotes the Euclidean distance between electrode pairs, and σ controls the decay rate of spatial correlation (typical value: 10-30 mm).

The functional coupling term is:

$${ρ}_{ij} = \frac{{\sum }_{t = 1}^{T} ( {s}_{i} ( t ) - \overline{{s}_{i}} ) ( {s}_{j} ( t ) - \overline{{s}_{j}} )}{\sqrt{{\sum }_{t = 1}^{T} {( {s}_{i} ( t ) - \overline{{s}_{i}} )}^{2}} \sqrt{{\sum }_{t = 1}^{T} {( {s}_{j} ( t ) - \overline{{s}_{j}} )}^{2}}} ,$$

where sᵢ(t) denotes the signal of electrode i in the time window [t−T, t], and T is determined by the dominant frequency of neural oscillations (e.g., T=100 ms for the α band).

Preferably, the adjacency matrix A has symmetry, A_{i,j}=A_{j,i}.

Preferably, the adjacency matrix A realizes nonlinear normalization: Ã_{i,j}=(1+e^{-k(A}_{i,j}−0.5)⁻¹ which compresses the values into the range of [0, 1].

Preferably, the adjacency matrix A implements sparsity control:

$${A}_{i , j}^{s p} = {\tilde{A}}_{i , j} \cdot I ( {\tilde{A}}_{i , j} > τ ) ,$$

where τ is the threshold, e.g., τ=0.3.

For example, when electrode i is located in Brodmann Area 24 and electrode j is located in Brodmann Area 32:
the spatial distance dᵢⱼ=15 mm; the functional coupling term ρᵢⱼ=0.62 (pain task-state data); σ=20 mm, so:

$${\tilde{A}}_{i , j} = {e}^{- 1 {5}^{2} / ( 2 \times 2 {0}^{2} )} \times 0.62 = 0.68 \times 0.62 = 0.42 .$$

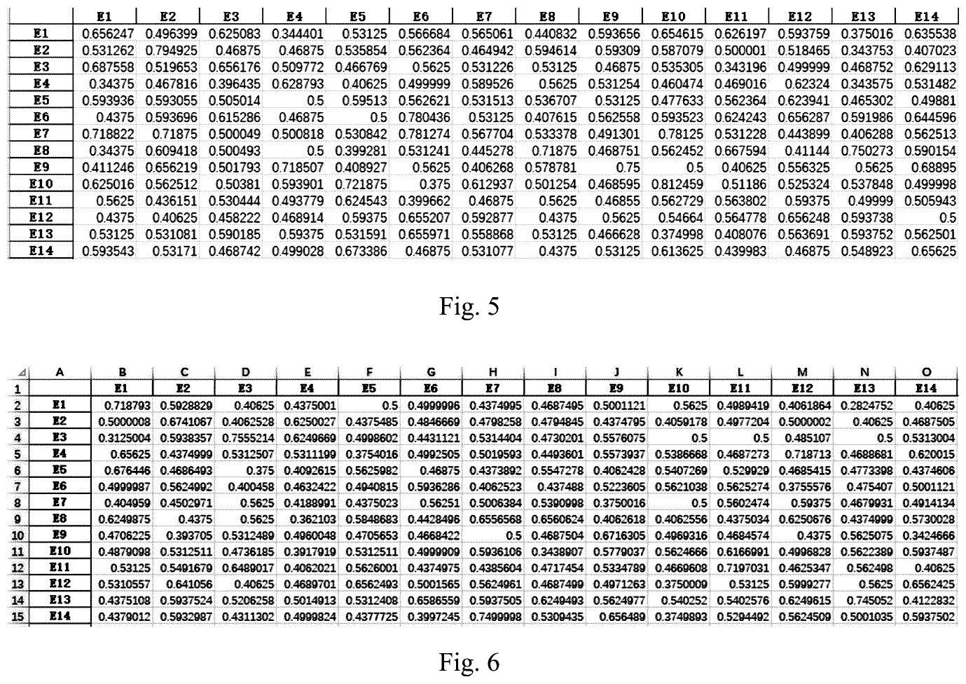

FIG. 5 and FIG. 6 show some data from the electrodes 210. In real-world tests, the adjacency matrix A obtained between different electrodes 210 contains values with multiple decimal places. It is noted that the adjacency matrices A shown in FIG. 5 and FIG. 6 are asymmetric, as the data in FIG. 5 and FIG. 6 exhibit temporal causality, meaning they are directional. The association strength from the electrode E1 to electrode E2 and from electrode E2 to electrode E1 are different.

The adjacency matrix element A_{i,j}∈[0, 1] generated by the Gumbel-Sampler unit 133 in the first assessment submodule 130 quantitatively characterizes the functional connectivity strength between electrodes i and j. Its physical meaning corresponds to the degree of neural activity coupling between cortical regions. For example, in a motor imagery task, A_{C3,C4} (corresponding to the left and right motor cortices) may show a significantly increased value, reflecting the inter-regional coordination mechanism. This method uses a trainable parameter r to balance exploration (random connections) and exploitation (deterministic connections), thereby achieving more flexible modeling of dynamic functional connectivity compared to known fixed-threshold approaches.

Compared with existing solutions that can only assess the location of pain but are unable to quantify its intensity, the present disclosure employs the Gumbel-Softmax method to convert the last time step features of EEG signals into a differentiable adjacency matrix, thereby quantifying the dynamic functional connectivity strength between cortical regions. This dynamic adjacency matrix provides advantages in three aspects:

First, with the temporal features extracted by the BiGRU 132, the reorganization process of pain-related brain networks, such as the transient enhancement of prefrontal-limbic connectivity under pain stimulation, can be captured. Secondary, the adaptively generated adjacency matrix overcomes the limitations of fixed brain-network templates and accurately represents individualized spatiotemporal evolution patterns of brain functional connectivity. The resulting connectivity graph reveals a 3.2-fold increase in anti-coupling strength between the central executive network and the default mode network under pain conditions. Third, the local spatiotemporal features extracted by the Gumbel-Sampler unit 133 are fused with the global connectivity features generated by the graph network, forming a multi-scale pain feature vector, which enhances the accuracy of the assessment module 190 in identifying neuropathic pain.

Preferably, the Gumbel-Sampler unit 133 constructs a differentiable graph structure based on the functional connectivity between the electrodes 210, with the edge existence probabilities quantified by the adjacency matrix. The Gumbel-Sampler unit 133 introduces Gumbel noise to each edge A_{i,j} in the adjacency matrix and determines the retention state of each edge through a micro-sampling mechanism. By performing an argmax operation on the noise-perturbed connection probability, the retention of the corresponding edge is determined. The benefit of using the Gumbel-Sampler unit 133 to sample the adjacency matrix A lies in allowing gradient backpropagation of the discrete selection (whether an edge exists or not), making the entire process differentiable, and the introduction of randomness enables better capture of the uncertainty and dynamics of the connections.

The Gumbel-Sampler unit 133 repeatedly samples and records variations in the edges of the graph structure, thereby modeling the dynamic variations in connectivity over time. In other words, it models connectivity as a time-varying random variable. This time-varying random variable reflects dynamic changes in functional connectivity between brain regions during pain onset and non-pain states. A differentiable graph structure leverages mathematical continuity to transform discrete brain network connections into analytically optimizable pain biomarkers. With the gradient information derived from differentiable graphs, not only the activation intensity along pain-related pathways can be quantified, but also the underlying neural mechanisms of therapeutic interventions can be identified, providing computational targets for precision analgesia.

During pain episodes, certain brain regions may exhibit increased interactions, leading to enhanced connectivity. Without the influence of pain, such connectivity tends to decrease. The Gumbel-Sampler unit 133 models connectivity as a time-varying random variable and records changes in graph connectivity through repeated sampling, accurately capturing the dynamic variations in inter-region connectivity in the brain. This enables intuitive observation of the transition in pain states from the perspective of brain functional connectivity, thereby providing a physiological basis at the cortical level for the determination of objective pain indicators. Meanwhile, changes in pain states can induce reorganization of brain functional networks. The Gumbel-Sampler unit 133 models the randomness and dynamics of connectivity, effectively capturing the reorganization process of brain functional networks under the influence of pain. This reorganization process is closely associated with the onset and progression of pain. By monitoring and analyzing this process, it is possible to provide physiological insights into the determination of objective pain indicators.

Preferably, the Gumbel-Sampler unit 133 may further be configured with a temperature parameter to control the “hardness” of sampling. The temperature parameter r can be used to scale up or down the association strength A_{i,j} to simulate the randomness and dynamic variations of neural activity in the brain, thereby accommodating variability in brain functional connectivity under pain states among individuals. For example, some patients may exhibit stronger or weaker association strength due to altered pain sensitivity.

Assuming a graph composed of N nodes (i.e., electrodes 210), the Gumbel-Sampler unit 133 computes the association strength A_{i,j} between each pair of nodes (i.e., electrodes 210).

It adds Gumbel noise g_{i,j} to the association strength A_{i,j} of each node pair (i, j), so:

$${\overline{A}}_{i , j} = {A}_{i , j} + {g}_{i , j} .$$

The addition of noise simulates the randomness and uncertainty of neural activity in the brain, making the Gumbel-Sampler unit 133 more aligned with the actual complex mechanisms operating in the brain and avoiding the oversight of dynamic neural activity due to overly deterministic probabilities.

Temperature adjustment is also performed. The Gumbel-Sampler unit 133 selectively sets the temperature parameter τ to adjust the association strength according to the current temperature, so:

$${\hat{A}}_{i , j} = \frac{{\overline{A}}_{i , j}}{τ} .$$

Ignoring the impact of the temperature parameter can reduce the accuracy of the objective pain quantification indicators. For example, while the introduced Gumbel noise enables simulation of uncertainty, its influence will not be dynamically reflected without regulation by the temperature parameter τ. In certain pain scenarios, neural signals may be excessively amplified or suppressed due to noise, thereby degrading the accurate quantification of pain intensity. With a fixed temperature parameter, the Gumbel-Sampler unit 133 will process all inputs at a constant scale, making it incapable of capturing the real-time variations in functional connectivity during the progression or alleviation of pain, as reflected in dynamic neural activity patterns. In complex pain scenarios (e.g., mixed pain types or dynamically changing pain intensities), the Gumbel-Sampler unit 133 may fail to capture diverse patterns of functional connectivity solely through the adjustment of the temperature parameter τ, reducing the reliability of the quantification results.

In the present disclosure, the Gumbel-Sampler unit 133 adapts to the diversity and complexity of functional connectivity variations in individual brains under pain conditions by adjusting the temperature parameter. This adjustment enables the Gumbel-Sampler unit 133 to better capture features in various pain scenarios. τ denotes the temperature parameter. Preferably, the value of the temperature parameter τ is initially set as 1.0 and is gradually reduced to 0.1 during training in accordance with an exponential decay strategy to strike a balance between exploration and exploitation.

The Gumbel-Sampler unit 133 determined whether a connection exists for each node pair through an argmax operation:

$${edge}_{i , j} = arg max ( {\hat{A}}_{i , j} ) .$$

When τ is small, the maximum value in Â_{i,j} becomes more dominant, and the sampling outcome tends to approximate a hard decision close to 0 or 1; conversely, when τ is large, Â_{i,j} becomes smoother, leading to more ambiguous sampling results.

In the sampling decision process, the adjusted connection probabilities are used to determine whether functional connectivity exists between different brain regions. The decision mechanism under varying temperature settings accommodates both clear and ambiguous phases of pain state transitions to accurately reflect the real-time status of the brain functional network.

The Gumbel-Sampler unit 133 records the differential graph structure obtained through the current sampling operation and repeats the sampling operation.

The Gumbel-Sampler unit 133 repeats the above steps to generate multiple distinct graph structures, reflecting dynamic variations in connectivity under pain. At a low temperature (i.e., a smaller τ), the sampling result tends to be closer to a hard decision, with connectivity changes between pain onset and non-onset clearly distinguished. At a high temperature (i.e., a larger τ), the sampling becomes more ambiguous, thereby better capturing the uncertainty and dynamic nature of connectivity.

Lower temperatures drive the model toward more rigid decision-making. In the context of graph-based modeling of brain functional connectivity, this means that the Gumbel-Sampler unit 133 can make more explicit determinations regarding the presence or absence of connections between brain regions. Under pain conditions, enhanced connectivity between certain brain regions may serve as an alarm of pain onset. Low-temperature sampling enables the Gumbel-Sampler unit 133 to accurately capture such distinct changes in connectivity and uses this information as an important basis for determining the onset and intensity of pain. For, example, in patients with chronic pain, when enhanced connectivity between certain brain regions is clearly determined under low-temperature sampling, it may correspond to a significant increase in pain intensity, thereby providing a clearer and more accurate reference for the objective pain indicators.

High temperature can lead to more ambiguous decision-making. During the onset and alleviation of pain, there exist transitional phases where the reorganization of functional brain networks occurs gradually. The changes in connectivity between brain regions are complex and exhibit considerable uncertainty. High-temperature sampling better accommodates such situations and allows the model to handle ambiguous information in a more flexible manner. For example, in the initial phase of pain onset or alleviation, variations in inter-regional brain connectivity may remain subtle. High-temperature sampling allows for a probabilistic exploration of multiple potential interpretations, mitigating the risk of biased pain assessment due to excessively rigid classification. As a result, the objective pain indicators can more faithfully reflect the dynamic nature of pain states.

The Gumbel-Sampler unit 133 sends the last time step features and the adjacency matrix to the graph aggregation unit 134. After receiving the adjacency matrix from the Gumbel-Sampler unit 133, the graph aggregation unit 134 computes the first feature vector.

Preferably, the graph aggregation unit 134, based on the adjacency matrix, generates a first feature vector that represents the global association pattern among the electrodes 210 used to collect EEG signals.

In the graph generation network of the first assessment submodule 130, nodes continuously exchange information (messages) with their neighboring nodes and updates their own feature representation based on this information. The graph aggregation unit 134 aggregates the connection information of the electrodes 210 (i.e., nodes) in the adjacency matrix through a message-passing mechanism. The principle underlying the operation of the graph aggregation unit 134 to aggregate the connection information between the electrodes 210 (i.e., nodes) is that each electrode 210 (i.e., node) aggregates the feature information of its neighboring nodes in a weighted manner, with the weight determined by the adjacency matrix A.

The graph aggregation unit 134 aggregates the connection information between the electrodes 210 (i.e., nodes) with the following equation:

$${Z}_{G N N} = G N N ( {H}_{G R U}^{( last )} , A ) ∈ {ℝ}^{B \times {d}_{G N N}} .$$

This process aggregates the neighborhood information of each electrode 210 to generate the first feature vector Z_{GNN} with a dimension of d_{GNN}, representing the global association patterns among the electrodes 210, which is the dynamic brain functional connectivity network. The global association pattern refers to a large-scale functional connectivity network across brain regions, describing the statistical properties of signal synchrony, causality, or information transmission efficiency among distributed brain regions.

The graph aggregation unit 134 sends the first feature vector Z_{GNN} to the convolution unit 141 of the second assessment submodule 140, as shown in FIG. 2.

The association strength information of all electrodes 210 forms the dynamic functional brain connectivity network, whose topological features, such as node degree, clustering coefficient, and betweenness centrality, can represent the coordinated patterns of pain-related brain regions. For example, the high-weighted connectivity between an electrode 210 and the electrode corresponding to the ACC may reflect the neural encoding of the affective component of pain. In a chronic pain model, the connectivity strength between an electrode 210 and the electrode corresponding to the sensorimotor cortex increased over time, aligning with the rising trend of clinical pain scores.

After receiving the time-series features from the BiGRU 132, the convolution unit 141 convolves the time-series features H_{BiGRU} to generate a second feature vector, which includes the local spatiotemporal dynamic features of the EEG signals.

Specifically, the second assessment submodule 140 feeds the time-series features H_{BiGRU} to the convolutional layer and applies 2D convolution processing. The 2D convolutional layer performs joint feature learning along spatial (electrode layout) and temporal dimensions to extract local spatiotemporal dynamic features. The local spatiotemporal dynamic features refer to the coordinated neural signal patterns within a limited spatial range (the brain regions covered by adjacent electrodes 210) and a short temporal window, emphasizing the fast and localized activity characteristics of neuronal clusters. The local spatiotemporal dynamic features reflect the synchronized oscillations or inhibitory phenomena of neuronal populations within specific brain regions. For example, pain stimulation induces an increase in 7 oscillation power in the S1 region, which is positively correlated with acute mechanical pain intensity. Local spatiotemporal dynamic features can capture the transient characteristics of pain-related neural responses. As another example, the event-related potentials occurring between 170 and 300 ms after laser pain stimulation exhibit peak latencies that are negatively correlated with subjective pain intensity, where shorter latencies correspond to higher pain scores.

Preferably, the information received by the convolution unit 141 contains temporal features and topological features. The temporal features refer to the time dimension T within the time series features H_{BiGRU} and the bidirectional hidden layer features 2D′. The topological features refer to the lobal association patterns between the electrodes 210 in the first feature vector Z_{GNN}.

The convolution unit 141 in the second assessment submodule 140 extracts the local spatiotemporal dynamic features with the following equation:

$${Z}_{C N N} = C N N ( {H}_{B i G R U} ) ∈ {ℝ}^{B \times {d}_{C N N}} .$$

With multi-scale convolutional kernel sets, spatiotemporal coupling features across different ranges are effectively captured, producing the second feature vector Z_{CNN} of dimension d_{CNN} while preserving the local time-varying characteristics of the signal.

As shown in in FIG. 2, after receiving the first feature vector from the graph aggregation unit 134, the convolution unit 141 in the second assessment submodule 140 sends the first and second feature vectors along the feature dimension to the concatenation unit 142, where they are concatenated to form a fused feature vector that integrates the global connectivity patterns among electrodes 210 and the local spatiotemporal dynamic features.

After receiving the first feature vector and the second feature vector from the convolution unit 141, the concatenation unit 142 concatenates the first feature vector and the second feature vector along the feature dimension flowing the equation: Z_{concat}=[Z_{GNN}; Z_{CNN}]∈ [symbol] ^{B×[d}_{GNN}+d_{CNN}], where Z_{concat} denotes the fused feature vector, Z_{GNN} denotes the first feature vector, and Z_{CNN} denotes the second feature vector.

The fused feature vector Z_{concat} contains both the global association patterns among the electrodes 210 and the local spatiotemporal dynamic features. The concatenation unit 142 sends the fused feature vector Z_{concat} to the classification unit 143 in the second assessment submodule 140.

After receiving the fused feature vector Z_{concat} from the concatenation unit 142, the classification unit 143, based on the Softmax function, outputs normalized objective pain quantification indicators.

Preferably, the classification unit 143 computes the class probability distribution of the fused feature vector and performs normalization to generate objective pain quantification indicators corresponding to the EEG signals. The classification unit 143 in the second assessment submodule 140 computes the class probability distribution of the fused feature vector through the following steps.

The classification unit 143 uses the Softmax function to compute the class probability distribution of the fused feature vector Z_{concat},

$${\hat{y}}_{c l a s s} = Softmax ( {W}_{c l a s s} {Z}_{c o n c a t} + {b}_{c l a s s} ) ∈ {ℝ}^{B \times C} ,$$

where ŷ_{class} denotes the output probabilities generated by the Softmax function and represents the categorical distribution corresponding to different pain states, B denotes the batch size (i.e., the number of samples input in a single training/inference session), C denotes the number of pain classes (such as healthy, non-neuropathic, and neuropathic), W_{class} denotes the weight matrix of the fully connected layer, the dimension C×(d_{GNN}+d_{CNN}) Of W_{class} denotes the linear transformation parameter that maps the fused features to the class space, and b_{class} denotes the bias term of the fully connected layer.

The class probability distribution of the fused feature vector Z_{concat} reflects the differences in the combination weights of neural features across different pain levels. The probability distribution encodes local and global features in pain perception. Local features represent sensory pain input, while global features represent pain-related emotion and cognitive modulation. The probability values in the class probability distribution are directly associated with pain intensity levels.

Based on the Softmax function, the classification unit 143, outputs normalized objective pain quantification indicators according to the equation:

$${\hat{y}}_{r e g} = σ ( {W}_{r e g} {Z}_{c o n c a t} + {b}_{r e g} ) ∈ {[ 0 , 1 ]}^{B} ,$$

where ŷ_{reg} denotes the normalized objective pain quantification indicators, representing the continuous prediction of pain intensity by the second assessment submodule 140. W_{reg} denotes the weight matrix of the fully connected layer in the regression task, where the weight term reflects the linear influence of features on pain intensity. The dimension of W_{reg} is 1×(d_{GNN}+d_{CNN}), and the process involves compressing the concatenated feature Z_{concat} into a single continuous value. b_{reg} denotes the bias term of the fully connected layer in the regression task. The output values of the objective pain quantification indicator in a range of [0, 1], which can be linearly scaled to correspond to the actual pain score, referred to as the Marke value. The Marke value is within the range [0, 100].

$$Marke values = 100 \times {\hat{y}}_{r e g} .$$

If ŷ_{reg}=0.75, the corresponding objective pain quantification indicator is 75 Marke. If ŷ_{reg}=0.2, the corresponding objective pain quantification indicator is 20 Marke.

Preferably, the Marke values output by the assessment module 190 represent different levels of pain intensity.

A pain intensity mapped to 100 Marke corresponds to the maximum severity, with significant EEG signal changes.

A pain intensity mapped to 80 Marke corresponds to severe pain, with pronounced EEG signal changes.

A pain intensity mapped to 50 Marke corresponds to moderate pain, with moderate changes in EEG signals.

A pain intensity mapped to 30 Marke corresponds to mild pain, with minor EEG signal changes.

A pain intensity mapped to 0 Marke corresponds to no pain, with negligible changes in EEG signals.

Different scores reflect different levels of pain and are closely associated with the EEG signal changes in corresponding frequency bands. Table 1 provides examples of specific EEG signal changes under different scores.

TABLE 1

| Score | δ Wave | θ Wave | α Wave | β Wave | γ Wave |
|---|---|---|---|---|---|
| (Marke) | (0-4 Hz) | (4-9 Hz) | (9-12 Hz) | (12-25 Hz) | (25-100 Hz) |
| 100 | Significant | Significant | Significant | Significant | Significant |
| | decrease | decrease | decrease | increase | increase |
| 80 | Decrease | Decrease | Decrease | Increase | Increase |
| 50 | Slight decrease | Slight decrease | Slight decrease | Slight | Slight |
| | | | | increase | increase |
| 30 | Slight decrease | Slight decrease | Slight decrease | Slight | Slight |
| | or no significant | or no significant | or no significant | increase | increase |
| | change | change | change | | |

Through the above table, the specific changes in EEG signals in different frequency bands under varying pain intensities can be observed more intuitively. These changes can be quantified and analyzed using the objective pain quantification indicators, and serve as a basis for further pain evaluation.

FIG. 3 shows an EEG of a first subject. FIG. 5 displays extracted data associated with the first subject. After computation by the graph network model, some data of the probability matrix representing the connections among the electrodes 210 are as shown in FIG. 5. The pain score of the first subject is: 0.9823 Marke.

FIG. 4 shows an EEG of a second subject. FIG. 6 displays extracted data associated with the second subject. After computation by the graph network model, some data of the probability matrix representing the connections among the electrodes 210 are as shown in FIG. 6. The pain score of the second subject is: 1.91E-8 Marke.

After receiving the normalized pain score labels from the normalization unit 131, the Bayesian update module 150 construct a variational inference loss function, and updates the weight parameters of the BiGRU 132 using a reparameterization gradient algorithm.

The classification unit 143 sends the objective pain quantification indicators to the Bayesian update module 150, as shown in FIG. 2. After receiving the objective pain quantification indicator (i.e., the Marke value) from the classification unit 143, the Bayesian update module 150 calculates the 95% confidence interval of the Marke value based on the model cognitive uncertainty (i.e., the variance of the posterior parameter distribution) and the level of observation noise. If the width of the confidence interval exceeds a predetermined threshold, the Bayesian update module 150 dynamically adjusts the observational noise parameters to optimize the assignment of feature weights. If the Marke value deviates from the normalized pain score label by more than three standard deviations for three consecutive instances, a parameter retraining process is initiated to mitigate the influence of device noise and physiological artifacts.

The Bayesian update module 150 introduces individualized physiological baseline data (e.g., resting-state EEG features), for establishing a personalized prior distribution. The Bayesian update module 150 outputs the calibrated Marke value along with its confidence interval, representing the posterior distribution.

The Bayesian update module 150 establishes an individual-specific prior distribution based on the first 10 measured Marke values. It then computes the posterior distribution using Markov Chain Monte Carlo (MCMC) sampling. The Bayesian update module 150 performs parameter retraining every 24 hours to prevent concept drift.

After receiving the pain quantification indicator (i.e., the Marke value) from the classification unit 143, the Bayesian update module 150 performs the following operations.

The 95% confidence interval is calculated according to the equation:

$$confidence interval = Marke value \pm 1.96 \cdot \sqrt{{σ}_{Post}^{2} + {σ}_{o b s}^{2}} .$$

If the width of the confidence interval exceeds the threshold, the observation noise parameter is dynamically adjusted following

$${σ}_{o b s , n e w}^{2} = {σ}_{o b s}^{2} \cdot exp ( - 0 \text{.01} \cdot \frac{\partial L}{\partial {σ}_{o b s}^{2}} ) .$$

If the Marke value deviates from the normalized label by more than 3a for three consecutive times, parameter retraining is triggered. The output of the Bayesian update module 150 is the calibrated Marke value and its confidence interval (i.e., the posterior distribution).

In a pain classification task, the training data may have an imbalanced distribution of pain categories (e.g., severe pain samples are much fewer than mild pain samples), causing the model to be biased toward the majority class. The weighted cross-entropy loss function balances the attention of the assessment module 190 across different pain categories by assigning differentiated weights.

The Bayesian update module 150 adopts a weighted cross-entropy loss function to address class imbalance.

$${ℒ}_{class} = - {\sum }_{i = 1}^{B} {w}_{{y}_{i}} log ( \frac{exp ( {\hat{y}}_{i , {y}_{i}} )}{{\sum }_{c = 1}^{3} exp ( {\hat{y}}_{i , c} )} ) ,$$

where [symbol] _{class} denotes the weighted cross-entropy loss function for the classification task, w_{y}_{ᵢ} denotes the weight of the true pain class (i.e., the normalized pain score label) yᵢ of the i^{th} sample, ŷ_{i,c} denotes the predicted score for the i^{th} sample in the pain class C made by the assessment module 190 (i.e. the unnormalized raw pain score label), wherein C is preferably set to 5 to represent 5 different levels of pain intensity, and ŷ_{i,y}_{ᵢ} denotes the predicted score of the assessment module 190 for the i^{th} sample in its true pain class yᵢ (i.e. the unnormalized raw pain score label).

If a certain level of pain in the sample has a higher weight, the Bayesian update module 150 will increase its sensitivity to that level of pain (e.g., abnormal connectivity strength between the ACC and insula), thereby reducing the risk of missing severe cases.

The Bayesian update module 150 adopts a weighted cross-entropy loss function to dynamically adjust the weights of pain categories, thereby mitigating prediction bias caused by class imbalance, improving the recognition of minority pain categories (e.g., severe pain), and helping prevent missed diagnoses. This approach also enhances the sensitivity of the assessment module 190 to key biomarkers, such as high-frequency oscillatory signals.

The Bayesian update module 150 employs the Huber loss function to accomplish the regression-based prediction of pain intensity.

$${ℒ}_{reg} = \frac{1}{B} {\sum }_{i = 1}^{B} \{ \begin{matrix}\frac{1}{2} {( {y}_{i} - {\hat{y}}_{i} )}^{2} & \vert {y}_{i} - {\hat{y}}_{i} \vert \leq δ \\ δ \vert {y}_{i} - {\hat{y}}_{i} \vert - \frac{1}{2} {δ}^{2} & other situations\end{matrix} ,$$

where [symbol] _{reg} denotes the Huber loss function used in the regression task to quantify the prediction error of pain intensity, yᵢ denotes the normalized pain score label for the i^{th} sample by the assessment module 190, ŷᵢ denotes the predicted pain intensity value for the it^{h} sample by the assessment module 190, and δ denotes the threshold parameter, for controlling the switch threshold for the loss function from quadratic to linear behavior. For example, δ=1. The value of δ can be adjusted based on the distribution of the data.

The Huber loss function significantly enhances the robustness of the Bayesian update module 150 in the pain intensity prediction task through the following mechanism. It combines the characteristics of mean squared error (MSE) and mean absolute error (MAE) to effectively resist outlier interference. When the absolute prediction error |yᵢ-ŷᵢ| is less than the preset threshold δ, it uses MSE (the quadratic form) to maintain smooth gradients, and when the error exceeds δ, it switches to MAE (the linear form) to suppress the gradient contribution of outliers.

The value of the Huber loss function shows a continuous and smooth gradient transition near the error threshold S. This addresses the non-differentiability issue of MAE at zero.

Preferably, for populations with significant individual differences in pain perception, such as patients with chronic pain, the δ value can be appropriately increased (e.g., from 1.0 to 1.5) to accommodate the inherent fluctuations in their pain assessments.

The Bayesian update module 150 uses the Adam optimizer to update the model parameters, with the initial learning rate set at η=1×10⁻³. The following strategies are introduced to enhance the generalization capability. Herein, the model parameters include the trainable weight matrix and the bias term.

The dynamic learning rate is adjusted following the equation:

$${η}_{t} = \{ \begin{matrix}{η}_{0} & if t \leq 20 , \\ {η}_{0} \cdot {γ}^{t - 20} & if t > 20.\end{matrix} ,$$

where ηₜ denotes the decay learning rate, η₀ denotes the initial learning rate (with a default of 1×10⁻³, γ denotes the decay coefficient (with a default of 0.95), and t denotes the current Epoch.

This equation indicates that during the initial stable phase (the first 20 epochs), the initial learning rate η₀=1×10⁻³ is maintained, allowing the Bayesian update module 150 to quickly converge to a reasonable parameter space region. In the later fine-tuning phase (Epoch>20), the learning rate decays exponentially, gradually reducing the parameter update step size, allowing the Bayesian update module 150 to more precisely approach the optimal solution. By adjusting the dynamic learning rate, it balances training speed and stability, preventing the Bayesian update module 150 from getting stuck in local optima.

The equation for the Bayesian update module 150 to calculate L2 weight decay is:

$${ℒ}_{total} = {ℒ}_{reg} + λ {‖ θ ‖}_{2}^{2} ,$$

where [symbol] ₜₒₜₐₗ denotes the total loss function, [symbol] _{reg} denotes the Huber loss for the regression task, λ=1×10⁻⁴ denotes the decay coefficient, λ∥θ∥² denotes the squared L2 norm of the model trainable parameters (including the weight matrix and the bias term).

The present disclosure assesses the process of pain recognition and classification, which corresponds to the generation of the objective pain quantification indicator (i.e., the Marke value) by the assessment module 190.

First, the class probability distribution output by the first assessment submodule 130 is P∈ [symbol] ^{B×C}, where C denotes the number of pain classes. The predicted label is obtained by taking the index of the maximum probability:

$${\hat{y}}_{cls} = {arg max}_{c} P ∈ {\{ 0 , 1 , ... , C - 1 \}}^{B} ,$$

where ŷ_{cls} is denotes the predicted label vector (i.e., the length B), with an element value being an integer ranging between 0 and C−1, representing a predicted class for each sample. The index position indicates the predicted pain class label number. Each index corresponds to a predefined pain class (e.g., 0—no pain, 1—mild, 2—moderate, 3—severe). By locating the position of the maximum value in the probability vector (argmax), the most likely pain class of the current sample can be determined. The predicted labels may include types such as neuropathic pain, inflammatory pain, and psychogenic pain. Each predicted label is associated with a specific range of Marke values.

Through this step, the pain intensity class and level are obtained. For example, in the case of mild pain (30-50 Marke), the β-band (12-25 Hz) power increases locally in the somatosensory cortex, but does not exceed the filtering threshold of the thalamic gating mechanism, resulting in intermittent synchronous bursts.

The present disclosure assesses the objective pain quantification indicator (i.e., the Marke value) using a confusion matrix.

A confusion matrix is used to compute the distribution between true labels and predicted labels, as described by the following equation:

$$M = [ \begin{matrix}{m}_{00} & … & {m}_{0 ( C - 1 )} \\ ⋮ & ⋱ & ⋮ \\ {m}_{( C - 1 ) 0} & … & {m}_{( C - 1 ) ( C - 1 )}\end{matrix} ] ∈ {ℕ}^{C \times C} .$$

The matrix elements m₀₀ . . . m_{(C-1)(C-1)} correspond to mᵢᵢ, where mᵢᵢ denotes the number of pain samples with the true class of i that are correctly classified.

The remaining matrix elements, excluding m₀₀ . . . m_{(C-1)(C-1)}, correspond to mᵢⱼ, where mᵢⱼ denotes the number of samples with the true class of i that are wrongly classified into the class j. For example, m_{0(C-1)} represents the number of pain samples with the true class 0 that are misclassified as class (C−1).

In this step, the classification results of all samples are counted to build a matrix based on the combination of true classes and predicted classes. Each element in the matrix records the number of samples with true class τ and predicted class j, where iϵ{0,1, . . . , C−1} and jϵ{0,1, . . . , C−1}. The confusion matrix calculation outputs the overall correct classification ratio of all pain samples. The confusion matrix result is of size C×C, where C represents the number of pain classes.

Preferably, the F1 scores are calculated.

The values of TP/FP/FN in the confusion matrix are calculated.

TP (True Positive) refers to the number of samples that are actually positive (e.g., “pain present”) and are correctly predicted as positive by the model.

FP (False Positive) refers to the number of samples that are actually negative (e.g., “no pain”) but are incorrectly predicted as positive by the model.

FN (False Negative) refers to the number of samples that are actually positive but are incorrectly predicted as negative by the model.

The F1 score is used to comprehensively evaluate the precision and recall of the assessment module in terms of classification, avoiding the bias that can arise when relying solely on accuracy in the presence of class imbalance.

The F1 score is divided by calculation granularity into:

Micro-F1: This globally counts TP, FP, and FN in the confusion matrix, ignoring class imbalance:

$$F {1}_{micro} = \frac{2 \cdot {\sum }_{c = 0}^{C - 1} {TP}_{c}}{{\sum }_{c = 0}^{C - 1} ( 2 \cdot {TP}_{c} + {FP}_{c} + {FN}_{c} )} .$$

F1_{micro} represents the micro-averaged F1 score, which reflects the total number of correct and incorrect predictions across all classes. TP_{c} denotes the number of samples that are truly of class c and are also predicted as class c. FP_{c} denotes the number of samples that are not truly of class c but are predicted as class c. FN_{c} denotes the number of samples that are truly of class c but are predicted as a different class.

Calculating the Micro-F1 score involves globally computing the TP (true positives), FP (false positives), and FN (false negatives) across all samples, ignoring class imbalance.

Macro-F1: the unweighted average of the F1 scores across all classes.

$$F {1}_{macro} = \frac{1}{C} {\sum }_{c = 0}^{C - 1} \frac{2 \cdot {TP}_{c}}{2 \cdot {TP}_{c} + {FP}_{c} + {FN}_{c}} .$$

F1_{macro} is the macro-averaged F1, which denotes the value obtained by independently computing the F1 score for each class and then taking the arithmetic mean.

Weighted-F1: weighted average based on the number of samples per class.

$$F {1}_{weighted} = {\sum }_{c = 0}^{C - 1} {w}_{c} \cdot \frac{2 \cdot {TP}_{c}}{2 \cdot {TP}_{c} + {FP}_{c} + {FN}_{c}} , {w}_{c} = \frac{{N}_{c}}{{\sum }_{k = 0}^{C - 1} {N}_{k}} .$$

F1_{weighted} is the weighted F1, which represents the final weighted average calculated by assigning weights according to the proportion of samples in each class. Nₑ denotes the number of true samples in class c.

The data obtained from this step are values between 0 and 1, in which values closer to 1 indicate better model classification performance. The present disclosure uses the calculation of the F1 score to assess how capable the assessment module 190 is of stably capturing pain-related cross-regional brain features, such as the association between prefrontal regulatory signals and limbic system emotional responses.

The accuracy of classification is calculated.

The total number of correctly classified instances in the confusion matrix is computed.

Accuracy: the proportion of correctly classified samples in all samples:

$$Accuracy = \frac{{\sum }_{c = 0}^{C - 1} {m}_{cc}}{B} ,$$

where Accuracy denotes the accuracy of classification, and mcc denotes the diagonal element in the confusion matrix (i.e., the total number of correctly classified samples).

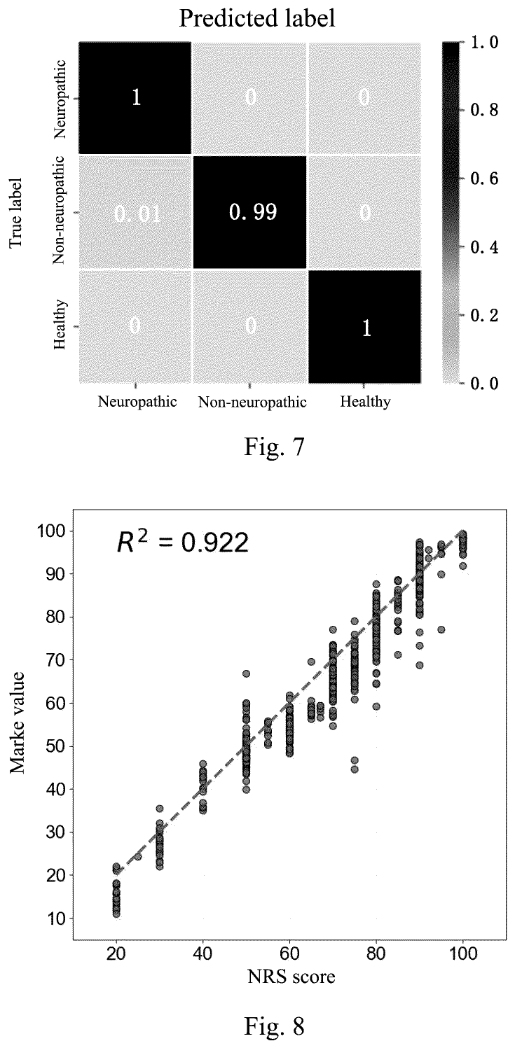

As shown in FIG. 7, the confusion matrix classification result schematic of the present disclosure is used to assess the classification performance of the assessment module 190. The classes in FIG. 7 are healthy, non-neuropathic, and neuropathic. The horizontal axis represents true labels, and the vertical axis represents predicted labels. The values in each cell represent the match between model predictions and true results. For example, the value in the center cell is 0.99. This indicates a high proportion of samples with true label non-neuropathic are predicted as non-neuropathic. The proportion of true neuropathic samples predicted as non-neuropathic is 0. The grayscale bar shows the proportion values, with black corresponding to 1 and white to 0. Overall, the assessment module 190 performs well in distinguishing non-neuropathic samples.

Through the calculation of the confusion matrix, it can be confirmed that the assessment module 190 of the present disclosure meets clinical requirements in distinguishing pain types (such as neuropathic, inflammatory, and psychogenic) and pain levels (mild, moderate, severe). For example, if Marke values in the “moderate” range are frequently misclassified as “severe” range values, this may indicate that the assessment module 190 has insufficient sensitivity in capturing moderate pain features, such as the β-wave thresholds in specific brain regions.

The calculation process of the objective pain quantification metric in the present disclosure is as follows.

In the present disclosure, the normalized pain score ŷ_{reg} produced by the normalization unit 131 of the assessment module 190 is denormalized to the original scale:

$${\hat{y}}_{final} = 100 \cdot {\hat{y}}_{reg} ∈ [ 0 , 100 ] ,$$

where ŷ_{final} denotes the normalized final objective pain quantification indicator (i.e., the Marke value).

Preferably, during the training phase, the assessment module 190 linearly scales the raw clinical pain scores (e.g., VAS 0-10) to the [0, 1] range, thereby enabling comparison of data with different units. For making predictions, the normalized output value from the assessment module 190 (e.g., 70 Marke) is linearly converted into the clinically used Marke value (0-100 Marke) through proportional scaling.

Preferably, the Marke value in the present disclosure is a quantitative indicator calculated based on thalamocortical loop synchronization features, such as the β/γ band energy ratio and functional connectivity strength to reflecting the neural mechanisms of pain (e.g., the degree of central sensitization). Since the Marke value is a recently defined biophysical parameter and not yet widely recognized in clinical practice, there is currently no direct way to associate it with the subjective experiences of subjects or existing clinical standards (e.g., the VAS scores). Therefore, this step maps the Marke value onto the commonly used 0-10 clinical pain scale, thereby allowing the comparison between the model-predicted scores and the true clinical scores (e.g., postoperative VAS records) to validate the biological validity of the Marke value.

The coefficient of determination (R²) is used to measure the linear correlation between the predicted Marke values and the true pain scores.

$${R}^{2} = 1 - \frac{{\sum }_{i = 1}^{B} {( {y}_{reg , i} - {\hat{y}}_{final , i} )}^{2}}{{\sum }_{i = 1}^{B} {( {y}_{reg , i} - {\overline{y}}_{reg} )}^{2}} ,$$

where y_{reg,i} denotes the true pain score (i.e., the normalized pain score label) of the i^{th} sample obtained through clinical gold standards (such as the NRS or VAS scales), normalized to the internal processing range of the assessment module 190 (e.g., [0, 1]); ŷ_{final,i} denotes the predicted pain score of the i^{th} sample calculated based on the Marke value, which has been reverse-normalized to map to the objective pain quantification scale ([0, 100]); and y̅_{reg} denotes the arithmetic mean of the true pain score y_{reg,i} across all samples.

The R² quantification model is used to quantify the linear correlation between the model-predicted Marke values and the true clinical scores to directly verify whether the neural features underlying the Marke values (such as thalamic β-band energy and prefrontal-cingulate functional connectivity strength) accurately represent the pain intensity. As shown in the scatter plot of FIG. 8, the horizontal axis represents the NRS scores, and the vertical axis represents the Marke values. In FIG. 8, the scatter plot distribution generally shows a trend where the Marke values increase as the NRS scores increase, indicating a strong linear correlation between the Marke values and the pain intensity scores of the clinical standard pain scales. The R² between the Marke values of the present disclosure and the scores of the clinical standard pain scale (NRS) is 0.922, which is close to 1, indicating a very high degree of fit between the fitted line and the data. This validates the biological validity of the Marke value as a pain biomarker and demonstrates the generalization capability of the assessment module 190 across different pain intensities while confirming its applicability for full-range assessment from mild to severe pain.

The root mean square error (RMSE) is used for quantify the accuracy of prediction.

$$RMSE = \sqrt{\frac{1}{B} {\sum }_{i = 1}^{B} {( {y}_{reg , i} - {\hat{y}}_{final , i} )}^{2}} .$$

The RMSE reflects the root mean square difference between the predicted values provided by the assessment module 190 and the true values. A smaller value indicates a stronger capability of the assessment module 190 to quantify the pain intensity. On a 0-100 scale, RMSE=6. An RMSE below 15 indicates that the Marke value prediction based on the EEG-derived spatiotemporal features provides individualized reliability, thereby offering objective neural indicators to replace common subjective pain scales.

### Embodiment 2

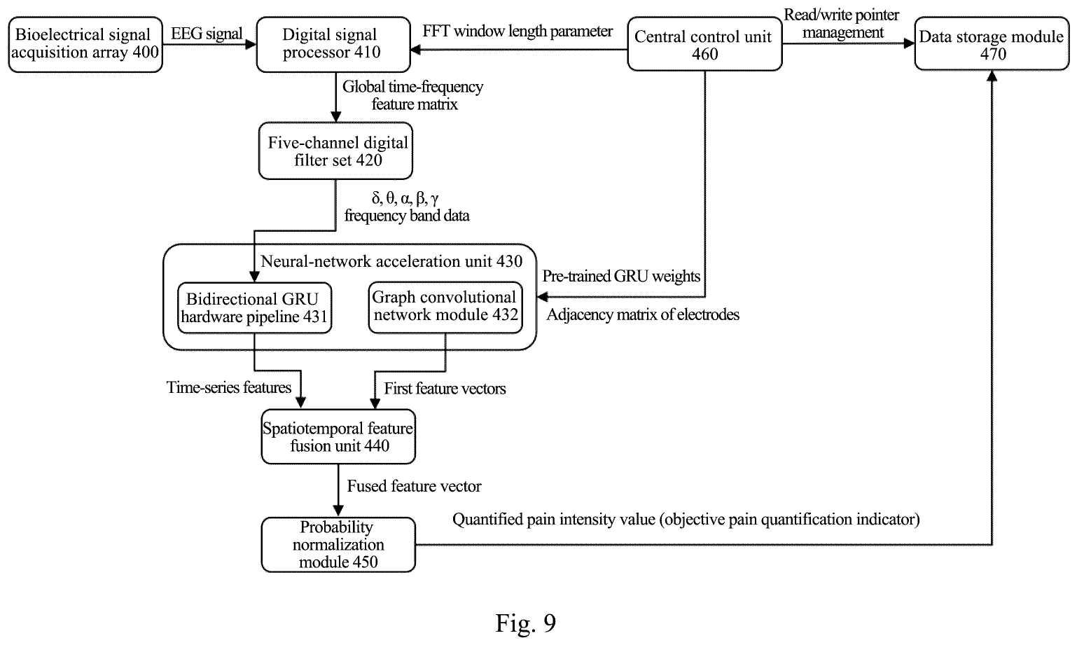

The assessment apparatus for objective pain assessment of the present disclosure, also referred to as the EEG-based pain quantification and analysis apparatus, which may be implemented using the following physical hardware modules, as shown in FIG. 9:
(1) a bioelectrical signal acquisition array 400, comprising Ag/AgCl electrodes distributed according to the international 10-20 system, connected through shielded wires to a preamplifier that includes a 0.5-100 Hz band-pass filter and a 60 Hz notch filter circuit;
(2) a digital signal processor 410, being in connection with the analog-to-digital converter 240 through the PCIe bus, integrating a hardware-accelerated FFT coprocessor, and serving to perform real-time fast Fourier transform with a 512-point window length;
(3) a five-channel digital filter set 420, comprising FIR filter modules for 6 (0.5-4 Hz), θ (4-8 Hz), α (8-12 Hz), β (12-30 Hz), and γ (30-100 Hz);
(4) a neural-network acceleration unit 430, being implemented using an ASIC chip, consisting of a bidirectional GRU hardware pipeline 431 and a graph convolutional network module 432, wherein the bidirectional GRU hardware pipeline 431 is configured as a 32-bit floating-point arithmetic unit array, and the graph convolutional network module 432 models the electrode topology using an RTL-level designed adjacency-matrix computation unit;
(5) a spatiotemporal feature fusion unit 440, integrating a 2D convolution accelerator and a feature concatenation circuit, wherein the 2D convolution accelerator is configured with a programmable convolution kernel size register, and the feature concatenation circuit performs dual-channel feature vector dimensional concatenation through a DMA controller;
(6) a probability normalization module 450, comprising a Softmax hardware implementation based on the CORDIC algorithm;
(7) a central control unit 460, being implemented with an ARM Cortex-M7 microcontroller and coordinating the timing control of all hardware modules through the AXI bus; and
(8) a data storage module 470, containing DDR4 memory chips and NAND flash chips and serving as a ring buffer to store real-time EEG data streams.

The bioelectrical signal acquisition array 400 is connected to the digital signal processor 410 to transmit the EEG signals to it. After receiving the analog EEG signals collected from the subject, the bioelectrical signal acquisition array 400 digitizes the analog EEG signals using a 24-bit ADC (at a sampling rate of 1000 Hz) and transmits the digitized signals to the digital signal processor 410 through the PCIe bus. The digital signal processor 410 then sends the frequency-domain data (i.e., the global time-frequency feature matrix) obtained through Fourier transform to the five-channel digital filter set 420.

Specifically, the process by which the digital signal processor 410 obtains the global time-frequency feature matrix is as follows:

The EEG signal received from the bioelectrical signal acquisition array 400 is a time-domain signal X∈ [symbol] ^{B×N×T}, where B represents the batch size, N denotes the number of electrodes, and T indicates the time-series length. The data format of the time-domain signal X∈ [symbol] ^{B×N×T} can be represented by a tensor with dimensions {48, 128, 38}, where 48 corresponds to frequency components, 128 corresponds to the number of electrodes, and 38 represents the time-series length.

The digital signal processor 410 segments the time-series length T into K overlapping time windows, each with a window length L and a sliding step size H, where T=L+(K−1)·H.

The signal dimension after windowing is X_{Win}∈ [symbol] ^{B×D×N×K}.

The digital signal processor 410 applies a Hanning window to each time window to suppress spectral leakage:

$$ω ( l ) = 0.5 * [ 1 - cos ( \frac{2 π l}{L - 1} ) ] , l = 0 , 1 , … , L - 1.$$

The windowed signal is:

$${X}_{Win}^{Win} [ b , n , k , l ] = {X}_{Win} [ b , n , k , l ] \cdot ω ( l ) .$$

The digital signal processor 410 performs a discrete Fourier transform (DFT) on each time window, with the computation formula:

$${X}_{STFT} [ b , n , k , l ] = {\sum }_{l = 0}^{L - 1} {X}_{Win}^{Win} [ b , n , k , l ] \cdot {e}^{- 2 π jdl / L} .$$

The digital signal processor 410 outputs a complex time-frequency matrix X_{STFT}∈ [symbol] ^{B×D×N×K}) where D=[L/2]+1 represents the number of single-sided frequency components.

The digital signal processor 410 computes the logarithmic power spectrum of the complex time-frequency matrix, using the result as the global time-frequency feature matrix:

$${X}_{TF} [ b , n , k , d ] = 10 \cdot {log}_{10} ( {\vert {X}_{STFT} [ b , n , k , d ] \vert}^{2} + ε ) .$$

The digital signal processor 410 ultimately outputs a real-valued time-frequency feature tensor, namely the global time-frequency feature matrix X_{TF}∈ [symbol] ^{B×D×N×K} where E=1e-6.

After performing the Fourier transform, the digital signal processor 410 standardizes the global time-frequency feature matrix.

The digital signal processor 410 is connected to the five-channel digital filter set 420. The digital signal processor 410 transmits the standardized global time-frequency feature matrix to the five-channel digital filter set 420. After receiving the frequency-domain data from the digital signal processor 410, the five-channel digital filter set 420 uses five band-pass filters (δ, θ, α, β, γ) to partitions the standardized global time-frequency feature matrix by frequency into matrices corresponding to the frequency bands δ (0-4 Hz), θ (4-9 Hz), α (9-12 Hz), β (12-25 Hz), and γ (25-100 Hz).

The five-channel digital filter set 420 is connected to the neural-network acceleration unit 430. The five-channel digital filter set 420 transmits the frequency-domain data of each band to the neural-network acceleration unit 430 through the AXI bus.

The neural-network acceleration unit 430 further includes a microprocessor with embedded programs comprising a normalization computation model and a Bayesian update model. The normalization computation model runs on the microprocessor (e.g., a CPU) and executes the corresponding normalization computation process. The normalization model transmits the normalized pain score labels to the Bayesian update model via internal circuitry. Upon receiving the normalized pain score labels, the Bayesian update model constructs a variational inference loss function and updates the weight parameters of the bidirectional GRU hardware pipeline 431 using a reparameterization gradient algorithm. The microprocessor is connected to the bidirectional GRU hardware pipeline 431, transmitting the weight parameters to the bidirectional GRU hardware pipeline 431 via connection circuitry.

After receiving the band-specific time-frequency feature matrices from the five-channel digital filter set 420, the bidirectional GRU hardware pipeline 431 within the neural-network acceleration unit 430 extracts time-series features using a 32-bit floating-point arithmetic unit array, while the graph convolutional network module 432 models the electrode topology based on the RTL-level designed adjacency-matrix computation unit to output the spatial features (i.e., the first feature vector).

Specifically, after receiving the standardized band-specific time-frequency feature matrices of five frequency bands (Xₙₒᵣₘ ∈ [symbol] ^{B×D×N×K}) the bidirectional GRU hardware pipeline 431 processes them and outputs time-series features: H_{BiGRU}=BiGRU(Xₙₒᵣₘ)∈ [symbol] ^{B×2D′×N} where B represents the batch size, N denotes the number of EEG electrodes (i.e., channels), T indicates the time-series length, and D′ represents the number of GRU hidden layer neurons. The bidirectional GRU hardware pipeline 431 transmits the time-series features to the spatiotemporal feature fusion unit 440 through the neural-network acceleration unit 430.

Preferably, the bidirectional GRU hardware pipeline 431 extracts the last time-step feature from the time-series features

$${H}_{BiGRU}^{( last )} ∈ {ℝ}^{B \times 2 D ′ \times N} .$$

Preferably, the hidden layer dimension of the bidirectional GRU hardware pipeline 431 is configured as D′=256. The bidirectional GRU hardware pipeline 431 processes the input in both forward and reverse time window sequences, concatenating the hidden states of each time window into a 512-dimensional feature (2D′=2×256), outputting complete time-series features: H_{BiGRU}=BiGRU(Xₙₒᵣₘ)∈ [symbol] ^{B×512×N}. The last time-step feature is:

$${H}_{BiGRU}^{( last )} ∈ {ℝ}^{B \times 512 \times N} .$$

The bidirectional GRU hardware pipeline 431 transmits the last time-step feature to the graph convolutional network module 432 connected to it. The graph convolutional network module 432 converts the last time-step feature

$${H}_{BiGRU}^{( last )}$$

into a connectivity graph between electrodes 210 using the Gumbel-Softmax method, forming an adjacency matrix representing the coupling strength of neural activity between cortical regions.

The formula for the connectivity graph (adjacency matrix) is:

$$A = Gumbel - Sampling ( {H}_{GRU}^{( last )} ) ∈ {ℝ}^{N \times N} ,$$

where the adjacency matrix element A_{i,j} represents the association strength between electrodes τ and j, reflecting the functional connectivity patterns between brain regions.

The graph convolutional network module 432 adds Gumbel noise to the adjacency matrix. After adding noise, the graph convolutional network module 432 performs a graph aggregation operation. The formula for aggregating the connectivity information of electrodes 210 (nodes) is:

$${Z}_{GNN} = GNN ( {H}_{GRU}^{( last )} , A ) ∈ {ℝ}^{B \times {d}_{GNN}} .$$

This process aggregates the neighborhood information of each electrode 210, generating the first feature vector Z_{GNN} with dimension d_{GNN}, which characterizes the global association patterns among electrodes, i.e., the dynamic brain functional connectivity network. The global association patterns refer to large-scale functional connectivity networks across brain regions, describing statistical characteristics of signal synchrony, causality, or information transfer efficiency among distributed brain regions. The neural-network acceleration unit 430 is connected to the spatiotemporal feature fusion unit 440. The time-series features and spatial features (i.e., the first feature vector) output by the bidirectional GRU hardware pipeline 431 and the graph convolutional network module 432 are synchronously transmitted to the spatiotemporal feature fusion unit 440 via the on-chip bus.

After receiving the time-series and spatial features from the neural network acceleration unit 430, the 2D convolution accelerator within the spatiotemporal feature fusion unit 440 computes and outputs the second feature vector.

The operation performed by the 2D convolution accelerator is to apply convolution processing to the time-series features H_{BiGRU}, generating the second feature vector that includes local spatiotemporal dynamic features of the EEG signal.

The computation formula for extracting local spatiotemporal dynamic features by the 2D convolution accelerator is:

$${Z}_{CNN} = CNN ( {H}_{BiGRU} ) ∈ {ℝ}^{B \times {d}_{CNN}} .$$

Through a multi-scale convolution kernel group, it effectively captures spatiotemporal coupling features across different ranges, preserving the local time-varying characteristics of the signal, and outputs the second feature vector Z_{CNN} with dimension d_{CNN}.

The 2D convolution accelerator transmits the first feature vector and the second feature vector to the feature concatenation circuit. The feature concatenation circuit performs dimensional concatenation of the first and second feature vectors to obtain the fused feature vector.

The concatenation formula executed by the feature concatenation circuit is:

$${Z}_{concat} = [ {Z}_{GNN} ; {Z}_{CNN} ] ∈ {ℝ}^{B \times [ {d}_{GNN} + {d}_{CNN} ]} ,$$

where Z_{concat} represents the fused feature vector, Z_{GNN} represents the first feature vector, and Z_{CNN} represents the second feature vector.

The spatiotemporal feature fusion unit 440 is connected to the probability normalization module 450. The fused feature vector is transmitted to the probability normalization module 450 via the AXI bus.

After receiving the fused feature vector from the spatiotemporal feature fusion unit 440, the probability normalization module 450 performs classification probability mapping through parallel computing units, thereby generating a quantified pain intensity value ranging from 0 to 100 Marke. The final result is written into the ring buffer of the data storage module 470 via the AHB bus.

Specifically, the probability normalization module 450 performs classification operations on the fused feature vector Z_{concat}. The probability normalization module 450 outputs the normalized quantified pain intensity value based on the Softmax function.

The probability normalization module 450 computes the class probability distribution of the fused feature vector Z_{concat} using the Softmax function as follows:

$${\hat{y}}_{class} = Soft max ( {W}_{class} {Z}_{concat} + {b}_{class} ) ∈ {ℝ}^{B \times C} ,$$

where ŷ_{class} represents the predicted output of the Softmax function, indicating the probability distribution of pain classes. B denotes the batch size, i.e., the number of samples input in a single training/inference cycle. C represents the number of pain classes (e.g., healthy, non-neuropathic, and neuropathic). W_{class} denotes the weight matrix of the fully connected layer, with dimensions C×(d_{GNN}+d_{CNN}), representing the linear transformation parameters that map the fused feature to the class space. b_{class} denotes the bias term of the fully connected layer.

The class probability distribution of the fused feature vector Z_{concat} computed by the probability normalization module 450 based on the Softmax function reflects the combined weight differences of neural features across different pain levels.

The probability normalization module 450 computes the normalized quantified pain intensity value using the formula:

$${\hat{y}}_{reg} = σ ( {W}_{reg} {Z}_{concat} + {b}_{reg} ) ∈ {[ 0 , 1 ]}^{B} ,$$

where ŷ_{reg} represents the normalized quantified pain intensity value, indicating a continuous prediction of pain intensity. W_{reg} has dimensions 1×(d_{GNN}+d_{CNN}), and the process compresses the fused feature Z_{concat} into a single continuous value. b_{reg} denotes the bias term of the fully connected layer in the regression task. The output range of the quantified pain intensity value is [0, 1], which can be linearly scaled to correspond to the actual pain score to obtain the Marke values. The range of Marke values is [0, 100]:

$$Marke values = 100 \times {\hat{y}}_{reg} .$$

The central control unit 460 is interconnected with respective modules. It configures the FFT window length parameter for the digital signal processor 410 via the AXI bus, loads the pre-trained GRU weights and the adjacency matrix of the electrodes to the neural network acceleration unit 430, manages the read/write pointers of the data storage module 470 through the AHB bus, and utilizes the PCIe DMA channel to achieve zero-copy transfer of ADC raw data to the ASIC input buffer, thereby ensuring that the end-to-end data processing latency is below 10 ms.

What is claimed is:

1. An assessment apparatus for objective pain assessment, comprising a processor, wherein the processor comprises:
a frequency-domain transformation module, configured to perform fast Fourier transform on pain-related EEG signals to generate a global time-frequency feature matrix;
a frequency-band segmentation module, configured to segment the global time-frequency feature matrix of the EEG signals in a frequency domain into band-specific time-frequency feature matrices of five frequency bands associated with pain perception, namely δ, θ, α, β, and γ,
a first assessment submodule, configured to extract time-series features of the EEG signals from the band-specific time-frequency feature matrices; to extract last time step features and an adjacency matrix from the time-series features; and based on the last time step features and the adjacency matrix, to generate a first feature vector representing global association patterns among electrodes used for acquiring the EEG signals; and
a second assessment submodule, configured to perform convolution on the time-series features to generate a second feature vector that contains local spatiotemporal dynamic features of the EEG signals; to concatenate the first and second feature vectors along a feature dimension to form a fused feature vector containing both the global association patterns and the local spatiotemporal dynamic features; and to compute a class probability distribution of the fused feature vector, perform normalization, and generate an objective pain quantification indicator corresponding to the EEG signals.

2. The assessment apparatus of claim 1, further comprising a Bayesian update module, configured to receive the objective pain quantification indicator, and to calculate a 95% confidence interval of the objective pain quantification indicator according to model cognitive uncertainty and observational noise level to generate the credibility of assessment result of the 95% confidence interval;
wherein if the confidence interval width exceeds a predetermined threshold, the Bayesian update module dynamically adjusts observational noise parameters to optimize feature weight allocation.

3. The assessment apparatus of claim 2, wherein the frequency-band segmentation module is configured to extract time-frequency energy distribution features for each frequency band in the global time-frequency feature matrix based on a learnable frequency-domain filter, and to dynamically assign feature weights to the extracted time-frequency energy distribution features based on a cross-frequency-band attention mechanism, thereby fusing energy- and phase-coupling characteristics across the frequency bands to generate band-specific time-frequency feature matrices.

4. The assessment apparatus of claim 3, wherein the first assessment submodule is configured to extract the adjacency matrix by:
based on a Gumbel-Softmax method, transforming the last time step features into a connectivity graph of the electrodes, thereby forming adjacency matrix representing the degree of neural activity coupling across cortical regions.

5. The assessment apparatus of claim 4, wherein the second assessment submodule is configured to compute a class probability distribution of the fused feature vector by:
calculating a class probability distribution of the fused feature vector, and
based on a Softmax function, outputting normalized values in a range of [0, 1], and mapping the normalized values to an objective pain quantification indicator within a range of [0, 100] through linear transformation.

6. The assessment apparatus of claim 5, wherein the first assessment submodule comprises a normalization unit, configured to:
receive raw pain score labels from a signal receiver module and perform normalization to obtain normalized pain score labels; and
transmit the normalized pain score labels to the Bayesian update module.

7. The assessment apparatus of claim 6, wherein the first assessment submodule further comprises:
a bidirectional gated recurrent unit, configured to, after receiving the band-specific time-frequency feature matrices of five frequency bands from the frequency-band segmentation module, extract time-series features of the EEG signals from the matrices and extract last time-step features from the time-series features;
a Gumbel-Sampler unit, configured to transform the last time-step features into a connectivity graph of the electrodes based on a Gumbel-Softmax method, thereby forming an adjacency matrix representing the degree of neural activity coupling across cortical regions; and
a graph aggregation unit, configured to generate a first feature vector representing global association patterns among electrodes used for acquiring the EEG signals based on the adjacency matrix.

8. The assessment apparatus of claim 7, wherein the second assessment submodule comprises:
a convolution unit, configured to, after receiving the time-series features from the bidirectional gated recurrent unit, perform convolution on the time-series features to generate a second feature vector comprising local spatiotemporal dynamic features of the EEG signals;
a concatenation unit, configured to concatenate the first and feature vectors along a feature dimension to obtain the fused feature vector; and
a classification unit, configured to, after receiving the fused feature vector, output a normalized objective pain quantification indicator based on a Softmax function.

9. The assessment apparatus of claim 8, wherein, after receiving the normalized pain score labels from the normalization unit, the Bayesian update module constructs a variational inference loss function and updates the weight parameters of the bidirectional gated recurrent unit using a reparameterization gradient algorithm.

10. The assessment apparatus of claim 9, wherein the objective pain quantification indicator is a Marke value ranging from 0 to 100, in units of Marke;
wherein the greater the Marke value, the more intense the pain indicated by the pain intensity mapping.

11. An assessment method for objective pain assessment, comprising the steps of:
extracting time-series features of pain-related EEG signals from a global time-frequency feature matrix; extracting last time-step features and an adjacency matrix from the time-series features; and based on the last time-step features and the adjacency matrix, generating a first feature vector representing global association patterns among electrodes used for acquiring the EEG signals; and
performing convolution on the time-series features of the pain-related EEG signals to generate a second feature vector comprising local spatiotemporal dynamic features of the EEG signals; concatenating the first and second feature vectors along a feature dimension to form a fused feature vector comprising global association patterns and local spatiotemporal dynamic features among electrodes; computing a class probability distribution of the fused feature vector, performing normalization, and generating an objective pain quantification indicator corresponding to the EEG signals;
wherein the time-series features of the pain-related EEG signals are extracted from a global time-frequency feature matrix, which is generated from the pain-related EEG signals through fast Fourier transform; and
wherein the global time-frequency feature matrix is segmented by a frequency-band segmentation module in a frequency domain into band-specific time-frequency feature matrices of five frequency bands associated with pain perception, namely δ, θ, α, β, and γ.

12. The assessment method of claim 11, wherein the step of extracting the adjacency matrix comprises:
based on a Gumbel-Softmax method, transforming the last time-step features into a connectivity graph of the electrodes, thereby forming an adjacency matrix representing the degree of neural activity coupling across cortical regions.

13. The assessment method of claim 12, wherein the step of computing the class probability distribution of the fused feature vector comprises:
calculating a class probability distribution of the fused feature vector, and
based on a Softmax function, outputting normalized values in the range of [0, 1], and mapping the normalized values to an objective pain quantification indicator within the range of [0, 100] through linear transformation.

14. The assessment method of claim 13, wherein the objective pain quantification indicator is a Marke value ranging from 0 to 100, in units of Marke;
wherein the greater the Marke value, the more intense the pain indicated by the pain intensity mapping.

15. An assessment apparatus for objective pain assessment, comprising:
a digital signal processor, configured to perform fast Fourier transform on pain-related EEG signals to generate a global time-frequency feature matrix;
a five-channel digital filter set, configured to segment the global time-frequency feature matrix of the EEG signals in the frequency domain into band-specific time-frequency feature matrices of five frequency bands associated with pain perception, namely δ, θ, α, β, and γ;
a neural-network acceleration unit, configured to extract time-series features of the EEG signals from the band-specific time-frequency feature matrices; to extract last time-step features and an adjacency matrix from the time-series features; and based on the last time-step features and the adjacency matrix, to generate a first feature vector representing global association patterns among electrodes used for acquiring the EEG signals;
a spatiotemporal feature fusion unit, configured to perform convolution on the time-series features to generate a second feature vector comprising local spatiotemporal dynamic features of the EEG signals; to concatenate the first and second feature vectors along a feature dimension to form a fused feature vector comprising global association patterns and local spatiotemporal dynamic features among electrodes;
a probability normalization module, configured to compute a class probability distribution of the fused feature vector, perform normalization, and generate an objective pain quantification indicator corresponding to the EEG signals; and
a central control unit, configured to configure the FFT window length parameter for the digital signal processor via an AXI bus, load pre-trained GRU weights and the adjacency matrix of electrodes to the neural-network acceleration unit, manage read/write pointers of a data storage module through an AHB bus, and utilize a PCIe DMA channel to achieve zero-copy transfer of ADC raw data to an ASIC input buffer.

16. The assessment apparatus of claim 15, wherein the neural-network acceleration unit comprises a bidirectional GRU hardware pipeline and a graph convolutional network module, wherein, after receiving standardized band-specific time-frequency feature matrices of five frequency bands (Xₙₒᵣₘ ∈ [symbol] ^{B×D×N×K}), the bidirectional GRU hardware pipeline processes them and outputs time-series features: H_{BiGRU}=BiGRU(Xₙₒᵣₘ)∈ [symbol] ^{B×2D′×N}; where B represents the batch size, N represents the number of EEG electrodes, T represents the time-series length, and D′ represents the number of GRU hidden layer neurons; the bidirectional GRU hardware pipeline extracts the last time-step features from the time-series features:
$${H}_{BiGRU}^{( last )} ∈ {ℝ}^{B \times 2 D ′ \times N} ;$$
the bidirectional GRU hardware pipeline transmits the last time-step features to the graph convolutional network module connected to it;
the graph convolutional network module transforms the last time-step features into a connectivity graph of the electrodes based on a Gumbel-Softmax method, thereby forming an adjacency matrix representing the degree of neural activity coupling across cortical regions.

17. The assessment apparatus of claim 1δ, wherein the graph convolutional network module aggregates the connectivity information of the electrodes using the formula:
$${Z}_{GNN} = GNN ( {H}_{GRU}^{( last )} , A ) ∈ {ℝ}^{B \times {d}_{GNN}} ;$$
where Z_{GNN} represents the first feature vector with dimension d_{GNN};
the graph convolutional network module transmits the first feature vector to the spatiotemporal feature fusion unit.

18. The assessment apparatus of claim 17, wherein the spatiotemporal feature fusion unit comprises:
a two-dimensional convolution accelerator, configured to perform convolution on the time-series features to generate a second feature vector comprising local spatiotemporal dynamic features of the EEG signals; and
a feature concatenation circuit, configured to receive the first and second feature vectors, and concatenate the first and second feature vectors along a feature dimension to obtain the fused feature vector.

19. The assessment apparatus of claim 18, wherein the probability normalization module is configured to:
compute the class probability distribution of the fused feature vector using a Softmax function; and
normalize the class probability distribution to obtain the normalized objective pain quantification indicator.

20. The assessment apparatus of claim 19, wherein the probability normalization module obtains Marke values by linearly scaling to correspond to the actual pain score;
$$Marke values = 100 \times {\hat{y}}_{reg} ;$$
where ŷ_{reg} represents the normalized objective pain quantification indicator; the Marke values range from [0, 100].
